SEQUENCE LISTING (10 sequences)

SEQ ID NO: 1 - DNA, 100 bp, Homo sapiens
gggcaaggtatggctgtgtacgttttgtgttacatttataagctggtgagattacggttcattttcatgtgaggcctggaggcaggagcaagatacttac

SEQ ID NO: 2 - DNA, 100 bp, Homo sapiens
gggcctgggatggggcttcactgctgtgacttcctcctgccaggggatttggggctttcttgaaagacagtccaagccctggataatgctttactttctg

SEQ ID NO: 3 - DNA, 100 bp, Homo sapiens
tgcagatcttcgtgaagaccctgactggtaagaccatcactctcgaagtggagccgagtgacaccattgagaatgtcaaggcaaagatccaagacaagga

SEQ ID NO: 4 - DNA, 100 bp, Homo sapiens
cggtcgtgatgtggtctgtggccaacgagcctgcgtcccacctagaatctgctggctactacttgaagatggtgatcgctcacaccaaatccttggaccc

SEQ ID NO: 5 - DNA, 20 bp, Artificial Sequence
  forward primer
acttaacgtgaggcgcaaaa

SEQ ID NO: 6 - DNA, 19 bp, Artificial Sequence
  Reverse primer
ctatctcggatggggatgg

SEQ ID NO: 7 - DNA, 20 bp, Artificial Sequence
  Forward primer
gacaagggaaggtggatgaa

SEQ ID NO: 8 - DNA, 20 bp, Artificial Sequence
  Reverse primer
ggggatgcttttaccagtga

SEQ ID NO: 9 - DNA, 20 bp, Artificial Sequence
  Forward primer
cacaattggccctggattag

SEQ ID NO: 10 - DNA, 20 bp, Artificial Sequence
  Reverse primer
tccctgtttcctctttgtgg

(19) United States
(12) Patent Application Publication
(10) Pub. No.: US 2021/0308077 A1
(43) Pub. Date: Oct. 7, 2021

(54) COMBINATIONS INCLUDING BETA-ADRENORECEPTOR AGONISTS FOR TREATMENT OF PARKINSON`S DISEASE AND MOVEMENT DISORDERS

(71) Applicant: The Brigham and Women`s Hospital, Inc., Boston, MA (US)

(72) Inventor: Clemens Scherzer, Boston, MA (US)

(21) Appl. No.: 16/606,534

(22) PCT Filed: Apr. 20, 2018
(86) PCT No.: PCT/US2018/028639

§ 371 Date: Oct. 18, 2019

Related U.S. Application Data
(60) Provisional application No. 62/487,541, filed on Apr. 20, 2017.

(51) Int. Cl. A61K 31/136; A61P 25/16; A61K 31/198; A61K 31/137; A61K 31/428; A61K 31/4704; A61K 31/403; A61K 31/166; A61K 31/138; A61K 31/4045; A61K 31/5377

(52) U.S. Cl. CPC A61K 31/136; A61P 25/16; A61K 31/198; A61K 31/137; A61K 31/428; A61K 31/5377; A61K 31/403; A61K 31/166; A61K 31/138; A61K 31/4045; A61K 31/4704

(57) ABSTRACT

Provided herein are methods of treating a subject who has a synucleinopathy (e.g., Parkinson's disease) that include: administering to a subject in need of such treatment therapeutically effective amounts of a β2-adrenoreceptor agonist and at least one therapeutic agent selected from the group consisting of: a synucleinopathy therapeutic agent, a β2-adrenoreceptor antagonist and a health supplement, wherein the health supplement is selected from the group consisting of caffeine, inosine, creatine, coenzyme Q10, vitamin E, and omega-3 fatty acids, to thereby treat Parkinson's disease in the subject.

### CLAIM OF PRIORITY

This application claims the benefit of U.S. Provisional Patent Application Ser. No. 62/487,541, filed on Apr. 20, 2017. The entire contents of the foregoing are hereby incorporated by reference.

### FEDERALLY SPONSORED RESEARCH OR DEVELOPMENT

This invention was made with Government support under Grant No. U01 NS082157, U01 NS 095736 and RO1 NS083845 awarded by the National Institutes of Health. The Government has certain rights in the invention.

### TECHNICAL FIELD

The present invention relates to the treatment of neurological disorders (e.g., Parkinson's disease) using drug combinations that include beta-adrenoreceptor agonists with other drugs and health supplements.

### BACKGROUND

Parkinson's disease is a movement disorder that affects one or more muscle groups. Symptoms of Parkinson's disease include: tremors, slowness of voluntary movements, change in gait, and unsteady balance. It is estimated that nearly 10 million people worldwide are living with Parkinson's disease.

### SUMMARY

The present invention is based, at least in part, on the discovery that the β2-adrenoreceptor regulates the transcription of α-synuclein, predicts the risk of Parkinson's disease in a ligand-specific fashion and constitutes a unique target for therapeutic intervention. Copy number mutations implicate excess production of α-synuclein as a possibly causative factor in Parkinson's disease (PD). Using an unbiased screen targeting endogenous gene expression, the β2-adrenoreceptor (β2AR) was discovered as a regulator of the α-synuclein gene (SNCA). β2AR ligands modulate SNCA transcription via histone 3 lysine 27 acetylation of its promoter and enhancers. During 11 years of follow up in four million Norwegians, the β2AR agonist salbutamol, a brain-penetrant asthma medication, was associated with reduced risk of developing PD (rate ratio =0.66, 95% C.I. 0.58-0.76). Conversely, a β2AR antagonist correlated with increased risk. β2AR activation protected model mice and patient-derived cells. Thus, β2AR links to transcription of α-synuclein and risk of PD in a ligand-specific fashion and constitutes a potential target for therapies.

Provided herein are methods of treating a subject who has a synucleinopathy that include: administering to a subject in need of such treatment therapeutically effective amounts of a β2-adrenoreceptor agonist and at least one therapeutic agent selected from the group consisting of: a synucleinopathy therapeutic agent, a β2-adrenoreceptor antagonist and a health supplement, wherein the health supplement is selected from the group consisting of caffeine, inosine, creatine, coenzyme Q10, vitamin E, and omega-3 fatty acids, to thereby treat Parkinson's disease in the subject.

In some embodiments of any of the methods described herein, the method further includes identifying the subject as having a synucleinopathy, e.g., Parkinson's disease prior to administering.

In some embodiments the method includes administering a β2-adrenoreceptor agonist, a synucleinopathy therapeutic agent and at least one of the health supplements.

In some embodiments, the β2-adrenoreceptor agonist is a blood brain penetrant β2-adrenoreceptor agonist. In some embodiments, the β2-adrenoreceptor agonist is selected from the group consisting of bitolterol, fenoterol, isoprenaline, levosalbutamol, orciprenaline, pirbuterol, procaterol, ritodrine, salbutamol, terbutaline, arformoterol, bambuterol, clenbuterol, formoterol, salmeterol, abediterol, carmoterol, indacaterol, olodaterol, vilanterol, metaproterenol, mabuterol, and zilpaterol. In some embodiments, the β2-adrenoreceptor agonist is selected from the group consisting of salbutamol, metaproterenol, clenbuterol and salbutamol.

In some embodiments, the synucleinopathytherapeutic agent is selected from the group consisting of levodopa, carbidopa, entacapone, ropinirole, rotigotine, pramipexole, bromocriptine, rasagiline, selegiline, amantadine and trihexphenidyl.

In some embodiments, the method includes administering a β2-adrenoreceptor agonist and a β2-adrenoreceptor antagonist.

In some embodiments, the β2-adrenoreceptor antagonist does not penetrate the blood brain barrier.

In some embodiments, the β2-adrenoreceptor antagonist is selected from the group consisting of carteolol, carvedilol, labetalol, nadolol, penbutolol, pindolol, sotalol, timolol, oxprenolol and butaxamine.

In some embodiments of any of the methods described herein, the method further includes administering therapeutically effective amounts of riluzole hydrochloride, or a pharmaceutically acceptable salt, prodrug, or isomer thereof.

In some embodiments, the β2-adrenoreceptor agonist and the at least one therapeutic agent are administered simultaneously to the subject; wherein the β2-adrenoreceptor agonist is administered to the subject prior to administration of the at least one therapeutic agent; or wherein the at least one therapeutic agent is administered to the subject prior to administration of the β2-adrenoreceptor agonist.

In some embodiments, the subject has Parkinson's disease.

In some embodiments, the subject does not have Parkinson's disease.

Provided herein are uses of a β2-adrenoreceptor agonist and a β2-adrenoreceptor antagonist in the manufacture of a medicament for treatment of a synucleinopathy.

Also provided herein are uses of a β2-adrenoreceptor agonist and a health supplement in the manufacture of a medicament for treatment of a synucleinopathy, wherein the health supplement is selected from the group consisting of caffeine, inosine, creatine, coenzyme Q10, vitamin E, and omega-3 fatty acids.

The term “therapeutically effective amount” refers to that amount of the β2-adrenoreceptor agonist and/or the therapeutic agent being administered sufficient to treat a synucleinopathy, e.g., Parkinson's disease. In one embodiment, the therapeutically effective amount is sufficient to prevent development of or alleviate to some extent one or more of the symptoms of the condition or disorder being treated, e.g., Parkinson's disease.

The term “subject” is defined herein to include animals, such as mammals, including but not limited to, primates (e.g., humans), cows, sheep, goats, horses, cats, rabbits, rats, mice, and the like. In preferred embodiments, the subject is a human. In some embodiments examples of any of the methods described herein, the subject is 40 years or older (e.g., 41 years old or older, 42 years old or older, 43 years old or older, 44 years old or older, 45 years old or older, 46 years old or older, 50 years old or older, 55 years old or older, 60 years old or older, 65 years old or older, 70 years old or older, 75 years old or older, 80 years old or older, 85 years old or older, 90 years old or older, or 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104 or 104 years old).

In some embodiments, the subject is a subject having a synucleinopathy, e.g., Parkinson's disease, suspected of having a synucleinopathy, e.g., Parkinson's disease or at increased risk of developing a synucleinopathy, e.g., Parkinson's disease (e.g., by virtue of family history, genetic testing, or presence of other identified risk factor).

In some embodiments, the subject does not present with a symptom (e.g., any of the symptoms described herein or known in the art) of a synucleinopathy neurological disorder (e.g., Parkinson's disease). In other embodiments, the subject has been diagnosed as having a synucleinopathy/neurological disorder (e.g., Parkinson's disease). In yet other embodiments, the subject has not been diagnosed as having a synucleinopathy disorder (e.g., Parkinson's disease).

In some embodiments, the subject has been diagnosed or identified as having a synucleinopathy neurological disorder (e.g., Parkinson's disease) that would benefit from treatment with a β2-adrenoreceptor agonist.

In some embodiments, the subject has previously been administered at least one dose of a therapeutic agent for a synucleinopathy, e.g., a Parkinson's therapeutic agent (e.g., any of the Parkinson's therapeutic agents described herein). In some embodiments, the subject is a participant in a clinical trial.

In other embodiments, the subject has been previously administered a different pharmaceutical composition and the different pharmaceutical composition was determined not to be therapeutically effective.

The term “population” when used before a noun means two or more the specific noun. For example, the phrase “a population of neuronal cells” means two or more neuronal cells.

The term “pharmaceutically acceptable salts” refers to salts of the active compounds that are prepared with relatively nontoxic acids or bases, depending on the particular substituents found on the compounds described herein.

As used herein, “obtain” or “obtaining” can be any means whereby one comes into possession of the sample by “direct” or “indirect” means. Directly obtaining a sample means performing a process (e.g., performing a physical method such as extraction or phlebotomy) to obtain a sample from the subject. Indirectly obtaining a sample refers to receiving the sample from another party or source (e.g., a third-party laboratory that directly acquired the sample). Thus, obtain is used to mean collection and/or removal of the sample from the subject. Some of the embodiments of any of the methods described herein can include obtaining a sample (e.g., a tissue biopsy) or samples from a subject.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosure will be apparent from the following detailed description and figures, and from the claims.

### SEQUENCE LISTING

The instant application contains a Sequence Listing which has been submitted electronically in ASCII format and is hereby incorporated by reference in its entirety. Said ASCII copy, created on Apr. 20, 2018, is named “Sequence Listing” and is 2,353 bytes in size.

### DESCRIPTION OF DRAWINGS

FIG. 1A is a schematic representation of a work flow leading to the identification beta-adrenoreceptor 2 (β2AR) as regulator of SNCA neuronal gene expression (top panel). “Campaign view” of compounds screened in the cell-based endogenous SNCA mRNA abundance (y-axis) observed in the drug-treated human neuroblastoma cells compared to DMSO-treated human neuroblastoma cells; housekeeping gene UBC was used to control for input RNA)

FIG. 1B shows the chemical structure, FDA approval, indication and blood-brain penetrance of three selective β2AR compounds.

FIG. 1C is a bar graph showing the relative endogenous Snca mRNA abundance in rat primary cortical neurons (n=4) after exposure to PAR agonists metaproterenol (5 μM) clenbuterol (20 μM) and salbutamol (10 μM).

FIG. 1D is a bar graph showing the relative endogenous α-Syn protein abundance in rat primary cortical neurons (n=4) after exposure to β2AR agonists metaproterenol (5 μM), clenbuterol (20 μM) and salbutamol (10 μM).

FIG. 1E is a bar graph showing the relative endogenous Snca mRNA abundance in a dose-dependent manner in neuroblastoma cells (N=6-8) after four days of clenbuterol treatment (5, 10, 20 μM). *P<0.05,**P<0.005, one-way ANOVA with Tukey's; means±standard error (SEM) are shown.

FIG. 1F is a bar graph showing the relative endogenous α-Syn protein abundance in a dose-dependent manner in neuroblastoma cells (N=6-8) after four days of clenbuterol treatment (5, 10, 20 μM). *P<0.05, **P<0.005, one-way ANOVA with Tukey's; means±SEM are shown.

FIG. 1G is a bar graph showing relative SNCA mRNA abundance in human SK-N-MC cells treated with β2-agonist metaproterenol (10 μM for 48 hours) at the screening, replication, and confirmation stages as compared as compared to vehicle control (P<0.005 in the confirmation stage) (n=8); P<0.005, two tailed Student's t-test; means±SEM.

FIG. 1H is a bar graph showing relative SNCA mRNA abundance in human SK-N-MC cells treated with metaproterenol (10 μM for 48 hours) using an independent digital gene expression platform (Nanostring) (P<0.005 in the confirmation stage) (n=8); P<0.005, two tailed Student's t-test; means±SEM.

FIG. 1I is a bar graph showing relative SNCA mRNA abundance in human SK-N-MC cells treated with β2-agonist clenbuterol (10 μM for 48 hours) at the screening, replication, and confirmation stages as compared as compared to vehicle control (P<0.005 in the confirmation stage) (n=8); P<0.005, two tailed Student's t-test; means±SEM.

FIG. 1J is a bar graph showing relative SNCA mRNA abundance in human SK-N-MC cells treated with β2-agonist sabutamol (10 μM for 48 hours) at the screening, replication, and confirmation stages as compared as compared to vehicle control (P<0.005 in the confirmation stage) (n=8); P<0.005, two tailed Student's t-test; means±SEM.

FIG. 1K is a bar graph showing relative SNCA mRNA abundance in human SK-N-MC cells treated with β2-agonist riluzole (10 μM for 48 hours) at the screening, replication, and confirmation stages as compared as compared to vehicle control (P<0.005 in the confirmation stage) (n=8); P<0.005, two tailed Student's t-test; means±SEM.

FIG. 2A is a bar graph showing cell viability in human SK-N-MC cells treated with metaproterenol (10 μM), clenbuterol (10 μM), or salbutamol (10 μM) (n=6-8); means±SEM.

FIG. 2B is a bar graph showing relative mRNA abundance of NEFL, RPL13 and GUSB in human SK-N-MC cells after 48 hours with metaproterenol (10 μM) (n=6-8); means±SEM.

FIG. 2C is a bar graph showing relative mRNA abundance of NEFL, RPL13 and GUSB in human SK-N-MC cells after 48 hours with clenbuterol (10 μM) (n=6-8); means±SEM.

FIG. 2D is a bar graph showing relative mRNA abundance of NEFL, RPL13 and GUSB in human SK-N-MC cells after 48 hours with salbutamol (10 μM) (n=6-8); means±SEM.

FIG. 3A is a bar graph showing relative ADRB2 mRNA abundance in human erythroleukemia HEL cells as compared to neuroblastoma cells. N=6-8; P<0.005, two tailed Student's t-test; means±SEM.

FIG. 3B is a bar graph showing relative SNCA mRNA abundance in human SH-SY5Y neuroblastoma cells as compared to human SK-N-MC cells. N=6-8; P<0.005, two tailed Student's t-test; means±SEM.

FIG. 3C is a bar graph showing relative SNCA mRNA abundance in HEL cells treated with metaproterenol (10 μM for 48 hours) or clenbuterol (20 μM for 48 hours). N=6-8; P<0.005, two tailed Student's t-test; means±SEM.

FIG. 3D is a bar graph showing relative SNCA mRNA abundance in SH-SY5Y cells treated with metaproterenol (10 μM for 48 hours) or clenbuterol (20 μM for 48 hours). N=6-8; P<0.005, two tailed Student's t-test; means±standard error (SEM).

FIG. 4A is a bar graph showing the relative Snca mRNA abundance in neuroblastoma cells (N=6-8) after two, three and four days of clenbuterol treatment (10 μM). *P<0.05, **P<0.005, one-way ANOVA with Tukey's; n=6-8; means±SEM.

FIG. 4B is a bar graph showing the relative α-Syn protein abundance in neuroblastoma cells (N=6-8) after two, three and four days of clenbuterol treatment (10 μM). *P<0.05, **P<0.005, one-way ANOVA with Tukey's; n=6-8; means±SEM.

FIG. 5A is a graph showing relative SNCA mRNA abundance in SK-N-MC cells treated with the indicated doses of metaproterenol after 48 hours. **P<0.005, Anova with Tukey's; n=4-6; means±SEM.

FIG. 5B is a graph showing relative SNCA mRNA abundance in SK-N-MC cells treated with the indicated doses of salbutamol after 48 hours. **P<0.005, Anova with Tukey's; n=4-6; means±SEM.

FIG. 6 shows representative immunoblots for endogenous α-synuclein protein abundance in substantia nigra homogenates of clenbuterol- and vehicle-treated mice (left panel), densitometric analysis of anti-α-synuclein immunosignal in clenbuterol treated mice relative to vehicle-treated mice (n=10 mice per group) using the anti-α-synuclein antibody 2F12.c) (middle panel), and densitometric analysis of anti-α-synuclein immunosignal in clenbuterol treated mice relative to vehicle-treated mice (n=10 mice per group) using the anti-α-synuclein antibody C-20) (right panel). *P<0.05, two-tailed Student's t-test; means±SEM.

FIG. 7A is a graph showing in vivo clenbuterol brain/plasma ratio (A, red line) and corresponding Snca mRNA levels in the substantia nigra (A; blue line) for 24 hours.

FIG. 7B is a graph showing the concentration of clenbuterol in brain (ng/g) for 24 hours.

FIG. 7C is a bar graph showing endogenous Snca expression in the PD-vulnerable substantia nigra of mice in the dose-finding mouse trial for 24 hours. *P<0.05, **P<0.005, two-tailed Student's t-test; means±SEM.

FIG. 7D is a bar graph showing endogenous Snca expression in the PD-vulnerable substantia nigra of mice in a controlled mouse trial for 24 hours. *P<0.05, **P<0.005, two-tailed Student's t-test; means±SEM.

FIG. 7E is a bar graph showing relative Snca mRNA expression in WT mouse primary neurons and in knock out β2AR gene (β2-AR KO) mouse primary neurons (n=6-9). *P<0.05, **P<0.005, two-tailed Student's t-test; means±SEM.

FIG. 7F is a bar graph showing relative α-Syn protein abundance in WT mouse primary neurons and in β2-AR KO mouse primary neurons (n=6-9). *P<0.05, **P<0.005, two-tailed Student's t-test; means±SEM.

FIG. 7G is a bar graph showing relative Snca mRNA expression in human SK-NM-C cells without or without silencing of β2ARs with RNA interference (n=3). *P<0.05, **P<0.005, two-tailed Student's t-test; means±SEM.

FIG. 7H is a bar graph showing relative α-Syn protein abundance in human SK-NM-C cells without or without silencing of β2ARs with RNA interference (n=3). *P<0.05, **P<0.005, two-tailed Student's t-test; means±SEM.

FIG. 7I is a bar graph showing relative Snca mRNA expression in human SK-NM-C cells treated with the β-blocker propranolol (n=8-12). *P<0.05,**P<0.005, two-tailed Student's t-test; means±SEM.

FIG. 7J is a bar graph showing relative α-Syn protein abundance in human SK-NM-C cells treated with the β-blocker propranolol (n=8-12). *P<0.05, **P<0.005, two-tailed Student's t-test; means±SEM.

FIG. 7K is a bar graph showing relative Snca mRNA expression in human SK-NM-C cells transiently transfected with ADRB2 constructs (n=6). *P<0.05, **P<0.005, two-tailed Student's t-test; means±SEM.

FIG. 7L is a bar graph showing relative α-Syn protein abundance in human SK-NM-C cells transiently transfected with ADRB2 constructs (n=6). *P<0.05, **P<0.005, one-way ANOVA with Tukey's test; means±SEM.

FIG. 7M is a bar graph showing relative α-Syn protein abundance in human SK-NM-C cells without or without silencing of β2ARs with RNA interference, or treated with clenbuterol (n=3). *P<0.05, **P<0.005, one-way ANOVA with Tukey's test; means±SEM.

FIG. 7N is a graph showing relative Snca mRNA expression in human SK-NM-C cells treated with metaproterenol, clenbuterol, salbutamol, metaproterenol+propranolol, clenbuterol+propranolol, salbutamol+propranolol (n=5-6) *P<0.05, **P<0.005, one-way ANOVA with Tukey's test; means±SEM.

FIG. 7O is a bar graph showing relative α-Syn protein abundance in human SK-NM-C cells treated with clenbuterol or clenbuterol+propranolol (n=8-12). *P<0.05, **P<0.005, one-way ANOVA with Tukey's test; means±SEM.

FIG. 7P is a bar graph showing relative α-Syn protein abundance in neuroblastoma cells treated with vehicle or clenbuterol (10 μM) for four days (n=6) as determined by a second ELISA using the anti-α-synuclein antibody Syn-1 as the detection antibody and the anti-α-synuclein antibody 1H9 as the capture antibody. *P<0.05, two-tailed Student's t-test; means±SEM.

FIG. 7Q is a bar graph showing relative α-Syn protein abundance in neuroblastoma cells treated with vehicle or propranolol (100 μM) for four days (n=6) as determined by a second ELISA using the anti-α-synuclein antibody Syn-1 as the detection antibody and the anti-α-synuclein antibody 1H9 as the capture antibody. *P<0.05, two-tailed Student's t-test; means±SEM.

FIG. 8A shows representative gene tracks of SNCA via H3K27 acetylation (H3K27ac) across the SNCA promoter and two enhancers in intron-4 (vertical bars 1, 2, and 3) (top panel). The SNCA gene, tracks for RefSeq transcripts, normalized reads density of RNA-seq in human brain (34), CAGE in human substantia nigra (10), histone modifications (H3K4me3, H3K4me1, and H3K27ac), and transcription factor occupancy (35). Bar graphs showing relative quantitative CHIP for H327Ac for Clenbuterol (blue), propranolol (orange), histone deacetylase inhibitor valproic acid (dark grey) and vehicle (grey) treatments across the three regulatory sites with P<0.05 by ANOVA with Tukey's; means±SEM of three independent experiments (bottom panel).

FIG. 8B is a bar graph showing relative SNCA mRNA levels (top panel) (n=7). *P<0.05, **P<0.005, one-way ANOVA with Tukey's; means±SEM, and immunoblots with an antibody against H3K27Ac (bottom panel).

FIG. 8C is a bar graph showing relative SNCA mRNA levels (top panel) (n=7) following co-treatment of clenbuterol with valproic acid. *P<0.05, two-tailed Student's t-test; means±SEM (n=4).

FIG. 9A is a graph showing the proportion of persons not developing Parkinson's disease (during 2008-2014) in the general population of Norway compared to individuals prescribed Salbutamol (during 2004-2007) at various doses. Cox's proportional hazard regression model adjusted for age, sex and level of education.

FIG. 9B is a graph showing the proportion of individuals not developing Parkinson's disease (during 2008-2014) in the general population compared to individuals (n=9,339) who had used at least 365 DDD of propranolol during 2004-2007. Cox's proportional hazard regression model adjusted for age, sex and level of education.

FIG. 9C are representative images illustrating TH+ neurons in the SNpc of MPTP- and MPTP+clenbuterol-treated animals.

FIG. 9D is a bar graph showing the number of TH+ neurons in SNc in a MPTP mouse model assayed by anti-TH immunostaining and stereology. n=6-8 animals per group; *P<0.05; **, P<0.01, one-way ANOVA with Tukey's; means±SEM, scale bar, 100 μm

FIG. 9E is a bar graph showing the number of TH+ neurons in SNc in a MPTP mouse model assayed by cresyl violet (CV) staining and stereology. n=6-8 animals per group; *P<0.05; **, P<0.01, one-way ANOVA with Tukey's; means±SEM, scale bar, 100 μm

FIG. 9F is a bar graph showing relative to SNCA mRNA expression (light blue, 3 days) and α-Syn protein expression (dark blue, 4 days) in Parkinson's patient induced pluripotent stem cells (iPSC)-derived neuronal precursor cells (NPCs) carrying the SNCA locus triplication after clenbuterol treatment (20 μM) (*P<0.05, * *P<0.005, two-tailed Student's t-test).

FIG. 9G is a bar graph showing levels of mitochondria-associated superoxide in NPCs carrying the SNCA triplication exposed to 20 μM rotenone and/or 20 μM clenbuterol for 18 hours (n=6). two-way ANOVA with Tukey's; *P<0.05; means±SD.

FIG. 9H is a bar graph showing cellular viability (based on resazurin, a fluorescent indicator dye of mitochondrial) of NPCs carrying the SNCA triplication in cells treated with 20 μM rotenone and/ or without 20 μM clenbuterol for 18 hours (n=6); two-way ANOVA with Tukey's; *P<0.05; means±SD.

FIG. 10 is a graph showing the proportion of individuals not having developed Parkinson's disease during 2008-2014 among persons on salbutamol during 2004-2007 (n=295,774) as compared to the general population of Norway. Cox's proportional hazard regression model adjusted for age, sex and level of education.

FIG. 11 are representative images illustrating cresyl violet positive cells in the SNpc region of vehicle or clenbuterol-treated C57bl/6 mice. Scale bar, 100 μm

FIG. 12 is a graph showing the relative SNCA mRNA abundance in human iPSC-derived neurons of a patient carrying the locus triplication after clenbuterol treatment (20 μM) for three days (*P<0.05, **P<0.005, two-tailed Student's t-tes; t; means±SEM).

FIG. 13 is a bar graph showing the relative SNCA mRNA abundance and protein abundance as determined by qPCR and ELISA, respectively, after 14 days of treatment with clenbuterol (10 μM) in SK-N-MC cells (**P<0.005, two-tailed Student's t-tes; t; n=6; means±SEM).

### DETAILED DESCRIPTION

The brains of most patients with Parkinson's disease (PD) are riddled with intracellular accumulations of α-synuclein protein known as Lewy bodies. Increasing dosage of the wild-type α-synuclein gene (SNCA) is sufficient to cause PD in kindreds carrying a locus multiplication (1, 2). In these patients, copies of functionally normal SNCA mRNA and protein are increased by about 50-100% (2, 3). Even smaller increases in α-synuclein transcription may play an analogous role in patients with sporadic disease carrying potential regulatory variants in this gene (4).

Traditionally, drug development in PD has focused on clearance of α-synuclein protein, on blockade of its transformation into toxic species, or amelioration of its downstream consequences. In contrast, we hypothesized that chemical compounds designed to reduce the transcription of the SNCA gene could allow one to prevent or slow down the disease process in selected patients, but this lacked a druggable target. Regulation of SNCA expression appears to include GATA transcription factor occupancy of evolutionarily conserved enhancers in intronic regions of SNCA (5), and possibly, the NGF- and bFGF-pathways (6), methylation (7), and microRNAs (8). However, none of these candidates can be easily targeted by currently available medicines. A high-throughput gene expression assay was developed for endogenous human SNCA expression in situ in neuronal cells. Human SK-N-MC neuroblastoma cells were cultured, drug-treated, and relative endogenous SNCA mRNA expression assayed in 384-well plates.

### Parkinson's Disease and Other Synucleinopathies

Parkinson's disease (PD) is a movement disorder that affects one or more muscle groups. Symptoms of Parkinson's disease include: tremors, slowness of voluntary movements, change in gait, and unsteady balance. It is estimated that nearly 10 million people worldwide are living with Parkinson's disease. See, e.g., Savitt et al., J Clin Invest. 2006 Jul. 3; 116(7): 1744-1754.

Although the present methods are exemplified with PD, one of skill in the art would understand that the methods can also be used to treat other synucleinopathies (neurodegenerative disorders characterized by the presence of increased levels, e.g., steady-state levels, of any one or more of soluble non-fibrillary variants, soluble oligomeric isoforms, insoluble non-fibrillary variants, complexes, and insoluble fibrillary aggregates of α-synuclein (αS) protein within cellular compartments of selective populations of neurons and glia. Thus, the αS steady-state level is understood to encompass all soluble as well as insoluble and intermediate (metastable) forms of the SNCA gene product). These can include, e.g., sporadic or heritable dementia with Lewy bodies (DLB); pure autonomic failure (PAF) with synuclein deposition; multiple system atrophy (MSA); hereditary neurodegeneration with brain iron accumulation; and incidental Lewy body disease of advanced age. In other embodiments, the synucleinopathy can be any one or more of: Alzheimer's disease of the Lewy body variant; Down's syndrome; progressive supranuclear palsy; essential tremor with Lewy bodies; familial parkinsonism with or without dementia; tau gene and progranulin gene-linked dementia with or without parkinsonism; Creutzfeldt Jakob disease; bovine spongiform encephalopathy; secondary Parkinson disease; parkinsonism resulting from neurotoxin exposure; drug-induced parkinsonism with α-synuclein deposition; sporadic or heritable spinocerebellar ataxia; amyotrophic lateral sclerosis (ALS); and idiopathic rapid eye movement sleep behavior disorder. In some embodiments, the subjects have, or do not have, a primary lysosomal storage disorder (LSD), such as Gaucher disease or Tay-Sachs disease. See, e.g., WO2008/144591. Methods of identifying subjects with these conditions are known in the art.

### Methods of Treatment

Provided herein are methods of treating synucleinopathies, e.g., Parkinson's disease, in a subject. Exemplary methods include administering to a subject in need of such treatment therapeutically effective amounts of a β2-adrenoreceptor agonist (BARA) and at least one (e.g., at least two, at least three, at least four, at least five, at least six, at least seven, at least eight, at least nine, or at least ten) therapeutic agent selected from the group consisting of: a Parkinson's disease therapeutic agent, a β2-adrenoreceptor antagonist and a health supplement, wherein the health supplement is selected from the group consisting of: caffeine, inosine, creatine, coenzyme Q10, vitamin E and fish oil (e.g., eicosapentaenoic acid (EPA), docosahexaenoic acid (DHA), omega-3 fatty acids), to thereby treat Parkinson's disease in the subject.

Also provided herein are methods of treating a subject that include: administering to a subject an isolated population of neuronal cells pre-treated with a β2-adrenoreceptor agonist; and administering to the subject at least one therapeutic agent selected from the group consisting of: a therapeutic agent for synucleinopathy, e.g., a Parkinson's disease therapeutic agent, a β2-adrenoreceptor antagonist and a health supplement, wherein the health supplement is selected from the group consisting of: caffeine, inosine, creatine, coenzyme Q10, vitamin E and fish oil (e.g., omega-3 fatty acids such as eicosapentaenoic acid (EPA) and/or docosahexaenoic acid (DHA)), to thereby treat the disease in the subject.

In some examples, the isolated population of neuronal cells is obtained from the subject who has Parkinson's disease. In some examples, the isolated population of neuronal cells includes triplication of the SNCA locus. In some examples, the isolated population of neuronal cells are inducible pluripotent stem cell (iPSC)-derived neuronal cells. In some embodiments of any of the methods of treatment described herein, the method further includes administering therapeutically effective amounts of riluzole hydrochloride, or a pharmaceutically acceptable salt, prodrug, or isomer thereof. Methods of making inducible pluripotent stem cells (iPSC) are known in the art. See, e.g., Singh et al. (2015) J Stem Cells 10(1): 43-62. Differentiation protocols of iPSC into neuronal cells are also known in the art and are provided in the Examples described herein.

In some instances, the subject having Parkinson's disease may have previously received a synucleinopathy disease therapeutic agent (e.g., any of the Parkinson's therapeutic agents described herein), or a treatment for another condition as described herein.

In some embodiments of any of the methods of treatment described herein, the method can result in a decreased risk of developing comorbidity in the subject (e.g., as compared to the risk of developing comorbidity in a subject having a similar neurological disorder (e.g., Parkinson's disease), but administered a different treatment).

In some embodiments of any of the methods of treatment described herein, the method can alleviate a negative side effect of any one of the BARA agent when administered alone (i.e., when the BARA agent is not administered in combination as described herein).

In some embodiments of any of the methods of treatment described herein, the method can result in increasing the life span of the subject (e.g., as compared to a subject having a similar synucleinopathy (e.g., Parkinson's disease), but administered a different treatment).

In some embodiments of any of the methods of treatment described herein, the method can result in an improvement in the movement and/or motor function of the subject (e.g., as compared to the movement and/or motor function of a subject having a similar synucleinopathy disorder (e.g., Parkinson's disease), but administered a different treatment).

Administering may be performed, e.g., at least once (e.g., at least 2-times, at least 3-times, at least 4-times, at least 5-times, at least 6-times, at least 7-times, at least 8-times, at least 9-times, at least 10-times, at least 11-times, at least 12-times, at least 13-times, or at least 14-times) a week. Also contemplated are monthly treatments, e.g. administering at least once per month for at least 1 month (e.g., at least two, three, four, five, or six or more months, e.g., 12 or more months), and yearly treatments (e.g., administration once a year for one or more years). Administration can be via any art-known means, e.g., intravenous, subcutaneous, intraperitoneal, oral, and/or rectal administration, or any combination of known administration methods.

Administration can include administering compositions in any useful format. For example, skilled practitioners will appreciate that a number of compositions are within the present invention. One useful composition may be a combination composition comprising a β2-adrenoreceptor agonist and at least one therapeutic agent selected from the group consisting of: a synucleinopathy (e.g., Parkinson's disease) therapeutic agent, a β2-adrenoreceptor antagonist and a health supplement, wherein the health supplement is selected from the group consisting of: caffeine, inosine, creatine, coenzyme Q10, vitamin E and fish oil (e.g., eicosapentaenoic acid (EPA), docosahexaenoic acid (DHA), omega-3 fatty acids). Such a combined composition can be administered to the subject in any useful dosing regimen. When using separate compositions, e.g., a first composition comprising a β2-adrenoreceptor agonist and a second composition comprising, e.g., a Parkinson's disease therapeutic agent, the compositions can be administered in any order. For example, the first composition can be administered before the second composition, the second composition can be administered before the first composition, or the first and the second compositions can be administered essentially simultaneously.

Skilled practitioners will appreciate that a subject can be diagnosed, e.g., by a medical professional, e.g., a physician or nurse (or veterinarian, as appropriate for the subject being diagnosed), as suffering from or at high risk (a risk significantly greater than the general population) for a synucleinopathy described herein, e.g., Parkinson's disease, using any method known in the art, e.g., by assessing a subject's medical history, performing diagnostic tests, and/or by employing imaging techniques.

Skilled practitioners will also appreciate that treatment need not be administered to a subject by the same individual who diagnosed the subject (or the same individual who prescribed the treatment for the subject). Treatment can be administered (and/or administration can be supervised), e.g., by the diagnosing and/or prescribing individual, and/or any other individual, including the subject her/himself (e.g., where the subject is capable of self-administration).

### Synucleinopathy/Parkinson's Disease Therapeutic Agents

A synucleinopathy therapeutic agent can be administered to the subject in methods described herein. The term “synucleinopathy therapeutic agent” refers to a therapeutic treatment that involves administering to a subject a therapeutic agent that is known to be useful in the treatment of a synucleinopathy, e.g., Parkinson's disease. For example, a Parkinson's disease therapeutic agent can increase and/or enhance the activity of dopamine in a subject. In other instances, a Parkinson's disease therapeutic agent can decrease the production of α-synuclein protein, reduce and/or inhibit the formation of Lewy bodies (i.e., accumulations of α-synuclein protein), inhibit the transformation of α-synuclein into toxic species.

Non-limiting examples of synucleinopathy therapeutic agents include: glucosylceramide synthase inhibitors (e.g., GZ667161), iron chelation agents, epigallocatechin gallate (EGCG), myeloperodixase inhibitors (e.g., AZD3241), affitopes (e.g., AFFITOPE PDO1A, AFFITOPE PD03A), and α-synuclein antibodies (e.g., PRX002, BIIB054).

Non-limiting examples of Parkinson's disease therapeutic agents include: levodopa, carbidopa, entacapone, ropinirole, rotigotine, pramipexole, bromocriptine, rasagiline, selegiline, amantadine and trihexphenidyl.

### B2-Adrenoreceptor Agonists

Provided herein are methods of treatment that include administering a β2-adrenoreceptor agonist. In some embodiments, the β2-adrenoreceptor agonist is a blood brain penetrant β2-adrenoreceptor agonist. Non-limiting examples of β2-adrenoreceptor agonist include: bitolterol, fenoterol, isoprenaline, levosalbutamol, orciprenaline, pirbuterol, procaterol, ritodrine, salbutamol, terbutaline, arformoterol, bambuterol, clenbuterol, formoterol, salmeterol, abediterol, carmoterol, indacaterol, olodaterol, vilanterol, metaproterenol, mabuterol, and zilpaterol. Various other β2-adrenoreceptor agonists are known in the art and are included herein.

### B2-Adrenoreceptor Antagonist

The β2-adrenoreceptor antagonist can be, e.g., a small molecule, an antibody, or an inhibitory nucleic acid (e.g., shRNA targeting α-synuclein, or miRNA targeting α-synuclein). In some instances, the β2-adrenoreceptor antagonist does not penetrate the blood brain barrier. Non-limiting examples of β2-adrenoreceptor antagonist include: carteolol, carvedilol, labetalol, nadolol, penbutolol, pindolol, sotalol, timolol, oxprenolol and butaxamine. Various other β2-adrenoreceptor antagonists are known in the art and are included herein.

### Pharmaceutical Compositions and Kits

Also provided herein are pharmaceutical compositions that include at least one (e.g., at least 2, 3, 4, 5, 6, 7, 8, 9, or 10) of any of the β2-adrenoreceptor agonists described herein and at least one (e.g., at least 2, 3, 4, 5, 6, 7, 8, 9, or 10) therapeutic agent, e.g., at least one β2-adrenoreceptor antagonist and/or health supplement, described herein.

The pharmaceutical compositions can be formulated in any matter known in the art. The pharmaceutical compositions are formulated to be compatible with their intended route of administration (e.g., intravenous, subcutaneous, intraperitoneal, rectal or oral). In some embodiments, the pharmaceutical compositions can include a pharmaceutically acceptable carrier (e.g., phosphate buffered saline). Single or multiple administrations of formulations can be given depending on for example: the dosage and frequency as required and tolerated by the patient. The dosage, frequency and timing required to effectively treat a subject may be influenced by the age of the subject, the general health of the subject, the severity of the disease, previous treatments, and the presence of comorbidities (e.g., cardiovascular disease, diabetes). The formulation should provide a sufficient quantity of active agent to effectively treat, prevent or ameliorate conditions, diseases or symptoms. Toxicity and therapeutic efficacy of compositions can be determined using conventional procedures in cell cultures, pre-clinical models (e.g., mice, rats or monkeys), and humans. Data obtained from in vitro assays and pre-clinical studies can be used to formulate the appropriate dosage of any composition described herein (e.g., any of the pharmaceutical compositions described herein).

Efficacy of any of the compositions described herein can be determined using methods known in the art, such as by the observation of the clinical signs of a Parkinson's (e.g., neurodegeneration, presence of Lewy bodies, tremors).

Also provided herein are kits that include at least one (e.g., at least 2, 3, 4, 5, 6, 7, 8, 9, or 10) of any of the β2-adrenoreceptor agonist described herein and at least one (e.g., at least 2, 3, 4, 5, 6, 7, 8, 9, or 10) of any of the therapeutic agents, e.g., at least one β2-adrenoreceptor antagonist, at least one Parkinson's disease therapeutic agent, and/or at least one health supplement, described herein. In some instances, the kits can include instructions for performing any of the methods described herein. In some embodiments, the kits can include at least one dose of any of the pharmaceutical compositions described herein. In some embodiments, the kits can provide a syringe for administering any of the pharmaceutical compositions described herein. The kits described herein are not so limited; other variations will be apparent to one of ordinary skill in the art.

### EXAMPLES

The disclosure is further described in the following examples, which do not limit the scope of the disclosure described in the claims.

### Materials and Methods

The following materials and methods were used in the examples set forth herein.

### Cell Culture and Drug Library

Human neuroblastoma SK-N-MC cells were acquired from American Type Culture Collection (ATCC). Cells were cultured at 37° C. in 5% CO₂ in DMEM/F12 supplemented with 10% fetal bovine serum (FBS) and 100 units per ml penicillin and streptomycin. The drug library was obtained from the Laboratory for Drug Discovery in Neurodegeneration (LDDN), Harvard NeuroDiscovery Center, Boston, Mass. The drug library consisted of approximately 1,126 small molecules. About 586 of the compounds were from Prestwick Chemical's (Illkirch, France) library, a unique collection of mostly off-patent small organic molecules, 90% being marketed drugs and 10% bioactive alkaloids or related substances. About 400 compounds were from the SPECS collection (available on the world wide web at.specs.net). 140 compounds generated in house by the LDDN were also included in the library. Compounds for the screening were stored as DMSO stocks. All drugs used for Confirmation Assay were purchased from Sigma (St. Louis, Mo.).

### High Throughput Screening (HTS).

### Screening Assay

Approximately 2×10⁴low passage SK-N-MC cells were seeded per well in 384-well cell culture plates for 24 hours. Compounds were then transferred into each well (final concentration 1 μM) by using a BioMekFX (Beckman Coulter) equipped with a 384-channel pipetting head. After 48 hours in cell culture incubator, cells were washed once in cold phosphate-buffered saline (PBS) by using BioTek ELx405 select CW plate washer and then lysed at room temperature (22-24° C.) with lysis buffer (Applied Biosystems) using the Cell-to-CT procedures according to the manufacturer's instructions (Applied Biosystems). After 5 min, lysis was terminated by incubation with stop solution (Applied Biosystems). 11 μL of each lysate were transferred to 384-well plates for RT in a total volume of 25 μl. 4 μL of RT reaction were then transferred to new 384-well plates for qPCR.

Using the Cell-to-CT kit (Applied Biosystems) we performed expression analysis directly from SK-N-MC neuroblastoma cells cultured in 384-well plates through in situ cDNA synthesis. In this miniaturized assay, high copy numbers of SNCA and of endogenous controls were detected as indicated by raw cycle threshold values of 25.11±0.02 (mean CT value±SEM) and 20.2±0.02 for SNCA and Ubiquitin C (UBC), respectively. Technical variation (using identical cDNA loaded in multiple wells) and biological variation (cDNA synthesized in situ in each of 384 wells of cultured SK-N-MC cells treated with DMSO) were low, with coefficients of variation of 1% and 5.2% respectively. At least ten DMSO-treated controls were included on each 384-well plate as negative external control and calibrator. An equal amount of cDNA derived from Universal Reference RNA was spotted on all plates as plate-to-plate control. Control reactions lacking template or reverse transcription were assayed for contamination with genomic DNA. Target and reference genes showed similar amplification efficiencies in a dilution series (Table 1).

TABLE 1
Target and Reference genes using in TaqMan assay

| Gene | Species | TaqMan Probe |
|---|---|---|
| Snca | Mouse | Mm01188700_m1 |
| Ubc | Mouse | Mm01198158_m1 |
| Rpl13 | Mouse | Mm02342645_g1 |
| Actb | Mouse | Mm00607939_s1 |
| Snca | Rat | Rn00569821_m1 |
| Ubc | Rat | Rn01499642_m1 |
| Rpl13 | Rat | Rn00821258_g1 |
| Actb | Rat | Rn00667869_m1 |
| SNCA | Human | Hs00240906_m1 |
| UBC | Human | Hs00824723_m1 |
| RPL13 | Human | Hs00761672_s1 |
| GUSB | Human | Hs99999908_m1 |
| ADRB2 | Human | Hs00196245_m1 |

### Threshold Considerations

Genomic duplications of SNCA are sufficient to cause an autosomal dominant form of PD and risk variants associated with sporadic Parkinson's disease might lead to a subtle increase in SAVA expression (1)(2). SNCA duplications are predicted to cause a 1.5-fold increase in SNCA expression (three SNCA locus copies in patients carrying one duplicated allele vs. two copies in the reference genome). GWAS variants have been associated with increased susceptibility for common, sporadic PD and with small increases in SNCA expression (4) that over time may lead to detrimental consequences for dopaminergic neurons. We hypothesized that a reduction of SNCA expression by 20-35% may be sufficient for helping to address the increased SNCA expression of SNCA duplication carriers and in patients with common PD carrying the SNCA susceptibility allele. Based on these considerations we required a reduction of SNCA expression levels of >35% in the Screening Assay and >20% in the Replication and Confirmation Assays. Secondly, we selected the ubiquitin gene UBC as reference gene to control for RNA loading in the Screening Assay based on low variation in expression <2% observed for this housekeeping gene in our preliminary studies of untreated and DMSO treated cells. Thirdly, we selected two additional, distinct housekeeping genes other than UBC, to control for RNA loading in the Replication and Confirmation Assays in order to identify compounds that robustly lower SNCA mRNA abundance independent of variation in housekeeping gene expression. Finally, we used a P value of ≤0.005 cutoff as an estimator of significance in the Confirmation Assay (adjusting the significance threshold for the number of ˜14 drug classes tested in the Replication Assay (represented by 41 compounds forwarded from Screening to Replication), e.g. 0.05/˜14≤0.005.

### Screening Assay: Detailed Threshold Criteria

Compounds were forwarded from the Screening Assay into the Replication Assay, if they met the following threshold criteria: 1, Compound lowered SNCA mRNA expression levels by >35% (fold change <0.65) compared to DMSO treated cells (fold changes were rounded to two decimal points) and 2, CT values of the first housekeeping gene UBC were within one standard deviation of the mean (in order to eliminate outliers and possible cytotoxic compounds). 1,126 compounds were evaluated in the Screening Assay; 61 of 1,126 were associated with a reduction in relative SNCA mRNA abundance with fold change <0.65 compared to vehicle treated cells; 26 of these 61 compounds showed changes in the housekeeping gene UBC of greater than one standard deviation from the mean and were thus excluded; the remaining 35 of 61 compounds were forwarded to the Replication Assay.

### Replication Assay and Detailed Threshold Criteria

41 compounds were forwarded into the Replication Assay. For each compound eight wells were treated with drug and compared to eight wells treated with DMSO alone. The Cell-to-CT procedure was employed using two new reference genes, ribosomal protein L13 (RPL13) and glucuronidase beta (GUSB) (instead of UBC) to control for input RNA. Compounds were forwarded from the Replication to the Confirmation Assay, if they met the following threshold criteria: 1, A compound reduced relative SNCA mRNA abundance using the second housekeeping gene RPL13 compared to vehicle treated control cells (as indicated by fold change <1.0); and 2, reduced relative SNCA mRNA abundance by 20% or more (fold change <0.80) compared to vehicle treated control cells using the third housekeeping gene GUSB. 10 compounds met these criteria and were forwarded from the Replication into the Confirmation Assay.

### Confirmation Assay and Detailed Threshold Criteria

The ten forwarded compounds were used in the Confirmation Assay. For each compound ten wells were treated with drug and compared to ten wells treated with DMSO alone. RNA was extracted using TRIzol® following the manufacturers protocol (Life Technologies). Freshly ordered compounds were used. Compounds were forwarded from the Confirmation to the ELISA Assay (see below), if they met they following significance criteria: 1, Compound reduced relative SNCA mRNA abundance by at least 20% (fold change <0.8) compared to vehicle treated controls and 2, with a p value ≤0.005. Six compounds met these criteria and were forwarded from the Confirmation Assay to the ELISA Assay stage (see below).

### NanoString Assay Performance

Probes were designed according to the manufacturer's design principles, including screening for inter- and intra-reporter and capture probe interactions, and selection for probes with optimal melting temperatures.

Target sequences information for NanoString probes:

SNCA:
(SEQ ID NO: 1)
GGGCAAGGTATGGCTGTGTACGTTTTGTGTTACATTTATAAGCTGGTGA
GATTACGGTTCATTTTCATGTGAGGCCTGGAGGCAGGAGCAAGATACTT
AC;
RPL13:
(SEQ ID NO: 2)
GGGCCTGGGATGGGGCTTCACTGCTGTGACTTCCTCCTGCCAGGGGATT
TGGGGCTTTCTTGAAAGACAGTCCAAGCCCTGGATAATGCTTTACTTTC
TG;
UBC:
(SEQ ID NO: 3)
TGCAGATCTTCGTGAAGACCCTGACTGGTAAGACCATCACTCTCGAAGT
GGAGCCGAGTGACACCATTGAGAATGTCAAGGCAAAGATCCAAGACAAG
GA
and
GUSB:
(SEQ ID NO: 4)
CGGTCGTGATGTGGTCTGTGGCCAACGAGCCTGCGTCCCACCTAGAAT
CTGCTGGCTACTACTTGAAGATGGTGATCGCTCACACCAAATCCTTGGA
CCC.

Direct counts of the target RNAs were measured in 125 ng of RNA by digital expression analysis based on NanoString technology (without reverse transcription into cDNA). Probes for the target and control RNA were multiplexed and assayed according to the manufacturer's protocol on the nCounter Digital Analyzer. The laboratory running the assay was blinded to the diagnosis. No-template (negative) controls containing water substituted for template were run and no signal was detected.

### Cell Viability Assay for Neuroblastoma Cells

CellTiter-Glo® Assay was used for measuring the cell viability in SK-NM-C cells. 100 μL CellTiter-Glo reagent (Promega) was added to 100 μL of cell culture medium per well in 96 well plates. Plates were agitated for 2 min and incubated for 10 min at room temperature (22-24° C.) before luminescence was measured.

### Cortical Neuronal Cultures

Primary cortical cultures were generated from E18 Sprague-Dawley rat embryos. The cortical region was dissected out in Hank's Balanced Salt Solution buffered with Hepes and dissociated with 0.125% trypsin (Invitrogen) for 17 min at 37 ° C., followed by trituration. Dissociated cells were plated at a density of 5×10⁵ cells/well or 2.5×10⁵ cells/well in 12-well or 24-well plates, respectively precoated with poly-D lysine (100 μg/mL). The cells were cultured in Neurobasal medium with B-27 supplement (Invitrogen) and glutamax and gentamycin. Half of the medium was changed every 4 days. Cortical neurons were treated with β₂AR agonists 10 days post plating. They were then harvested 48 hours post-treatment for mRNA extraction and 72 hours post-treatment for protein extraction.

### Hippocampal Neuronal Cultures

Primary hippocampal cultures were generated from day 0 neonatal wild type and β₂AR KO mice. The hippocampal neurons were plated on poly-D lysine (100 μg/mL) precoated plates and cultured in Neurobasal media with B-27 supplement and L-glutamine for 14 days. Post 14 days after plating the cells were harvested for mRNA and protein extraction.

### Human Pluripotent Stem Cell Culture

The derivation and culture of the WIBR-IPS-αSyn^{TRPL}line has been described previously (27).

### Human neural induction by embryoid body (EB) formation

To initiate differentiation, iPSC colonies were pretreated for 30-60 min with 5 μM Y-27632, a ROCK inhibitor (Calbiochem). They were single cell-dissociated after 5-10 minute exposure to accutase (StemProAccutase; Life Technologies) and then re-suspended in neural base (NB) medium, which is DMEM/F12 (Gibco/Life Technologies) supplemented with 0.5% N2 and 1% B27 (Life Technologies). Cells were plated in AggreWell 800 microwells (StemCell Technologies; priming and plating per manufacturer's protocol; 2.4×10⁶ cells were well) in NB medium supplemented with dual SMAD inhibitors (recombinant human Noggin (R&D Systems) at 200 ng/mL and 10 μM SB431542 (Tocris Bioscience), as well as 5 μM Y-27632. Noggin and SB431542 remained in the medium at these concentrations throughout the neural differentiation protocol.

On day 1 medium was half-changed. By day 2, well-formed neuralized EBs (NEBs) were typically observed in the AggreWells and transferred to Petri dishes (4 AggreWell wells/Petri dish) overnight, in NB medium. On day 4, NEBs were transferred to a dish coated with growth factor-reduced Matrigel (1:30 in DMEM: F12; BD Biosciences) for attachment. Y-27632 was omitted from this day until day 10. From day 5 to day 10, attached NEBs were additionally exposed to 20 ng/mL FGF2 (R&D Systems) and recombinant human Dkkl at 200 ng/mL (R&D Systems). On day 10, neural rosettes were dissected (P20 pipette tip), incubated in accutase supplemented with Dnasel (Sigma Aldrich) for 10 min at 37° C., and gently dissociated to small cellular clumps and single cells. After washing, the rosettes were re-plated on plastic dishes pre-coated with poly-L-ornithine and laminin (BD Biocoat) at high density (200,000/cm²) in neural progenitor cell (NPC) medium, which is NB medium supplemented with 20 ng/mL FGF2 (Life Technologies), supplemented overnight with 10 μM Y-27632. Typically, one Aggrewell 800 well provided sufficient NPCs for one to two 6-well plates at passage 0.

Thereafter, the surviving NPCs proliferated. Medium was changed daily. NPCs could be passaged up to 10 times before neural differentiation, and could be successfully freeze/thawed at early passage (p1 to p5) without compromising differentiation potential. The freezing medium used was NPC medium with 10% FBS (Hyclone).

### Human Cortical Neural Differentiation

To begin neural differentiation, NPCs were dissociated with accutase and re-plated on matrigel-coated T75 flasks (CytoOne). The next day medium was fully changed to Neural Differentiation (ND) medium, which is NB medium supplemented with recombinant human BDNF and GNDF (both at 10 ng/mL; R&D Systems) and dibutyryl cyclic AMP (500 μM; Sigma), and without FGF-2. Thereafter, media was half-changed every other day. On day 7-9, differentiating neurons were gently dissociated to single cell suspension and then resuspended in pre-chilled Hank's balanced salt solution (HBSS; Gibco/Life Technologies) supplemented with 0.1% bovine serum albumin (Gibco/Life Technologies). After a wash step, cells were plated on 6- or 24-well plates pre-coated with poly-ornithine and laminin (BD Biocoat). For maximum survival, 5 μm Y-27632 was used in the initial plating medium and cells were plated at high density (500,000-1×10⁶ cells/cm²). Medium was half-changed every 3 days for up to 8 weeks. After 8 weeks, cells were exposed to compound for 3 days, with a complete medium change every other day. The current data correspond to a single representative experiment with 6 technical replicates, but the experiment was repeated twice, each time giving a similar result.

### Pharmacokinetics Mouse Study

The clenbuterol pharmacokinetic (PK) study was done in wild-type C57BL/6J male mice by ChemPartner Co., LTD in Shanghai. The study was done with 4 groups of four mice; one control group and three treatment groups with clenbuterol administered via intraperitoneal injection at dosages of 1, 5 and 10 mg/kg once every 24 hours. Following completion of the administration schedule, blood samples were collected at various time points (e.g. 0.25, 2, 8, and 24 hours), from the four mice per group per time point (final time point of 24 hours post injection). Mice were sacrificed following each blood collection and brains were removed and snap frozen. Substantia nigra was utilized for SNCA mRNA and protein analysis, whereas the other portions were used for PK studies. Plasma and brain samples were analyzed by LC-MS/MS for compound concentration at each of three concentrations and at each time point. Standard pharmacokinetic parameters (e.g. AUClast, AUCINF, terminal elimination half-life (t_{1/2}), clearance, volume of distribution, time at maximum observed plasma concentration, etc.) were determined in both plasma and brain tissueby WinNonlin V 6.2 statistics software (Pharsight Corporation, California, USA) using a non-compartmental model.

### Placebo-Controlled Trial in Wild-Type Mice

Twenty C57BL/6J male mice (8 weeks old) (Jackson Laboratory, Bar Harbor Me.) were used for the placebo-controlled trial. Mice were exposed to a 12-hour light and 12-hour dark cycle, maintained at a constant temperature of 22° C., and were cared for in accordance with the National Institutes of Health Guide for the Care and Use of Laboratory Animals. Clenbuterol was purchased from Sigma (St. Louis, Mo.). Clenbuterol (10 mg/kg, injection volume 10 ml/kg) dissolved in saline was administered to mice (N=10) intraperitoneally. Saline was administered as a vehicle treatment (N=10). Mice were euthanized by CO₂24 hours post-injection. Brains were removed and snap frozen immediately in dry ice. Further dissections were performed on the frozen tissue to obtain substantia nigra regions. Tissue was then stored at −80° C. until mRNA or protein extraction was performed.

### Controlled Trial in the MPTP Mouse Model

Male C57bl/6 mice (8-10 weeks old) were analyzed. N-methyl-4-phenyl-1,2,3,6-tetrahydropyridine (MPTP-HCl; 20 mg/kg, measured as free base; Sigma, St. Louis, Mo.) was administered to mice intraperitoneally once per day for five consecutive days similar to described previously (26,36). Eight MPTP-treated mice assigned to the clenbuterol treatment group received one 10 mg/kg i.p. injection of clenbuterol (two days prior to MPTP treatment start) and then clenbuterol via oral gavage (10 mg/kg) daily for two weeks (starting on the day of the first MPTP treatment). Another eight MPTP-treated mice assigned to the vehicle treatment group received only oral drinking water (without clenbuterol). Additional control mice received i.p. injections of equivalent volumes of saline (0.9%) instead of MPTP intraperitoneally once per day for 5 consecutive days; of these eight received drinking water alone (without clenbuterol) and another eight received oral clenbuterol. Mice were sacrificed 14 days after initial MPTP/saline i.p. treatment.

After overnight postfixation in 4% paraformaldehyde, brains were cryoprotected in 30% sucrose before cryosectioning into 40 μM (used for stereology) free-floating sections, obtained as described previously (36). Immunostaining was performed as described previously (36) using mouse anti-tyrosine hydroxylase (Abcam; 1:700) and diaminobenzidine (DAB). TH+ cells were analyzed by sterological estimates as described previously (36). Briefly, the total number of TH+neurons in the SNc was calculated by optical fractionation using Stereo Investigator (version 6; MicroBrightField, Williston, Vt.). 40 μM of brain sections were examined from bregma −2.54 to −3.88 of the SNc. For each brain, six coronal sections were examined using a 100× lens. Total number of TH+ neurons was determined using the optical fractionator. Adjacent tissue was also stained using cresyl violet (Sigma, 0.01% for 5 min) to confirm cell loss as assessed by TH analysis.

### siRNA Transfection

SK-N-MC cells grown in 6-well dishes at 40% confluence were transfected with SMARTpool: ON-TARGET plus ADRB2 siRNA (cat no. L-005426-01-0005, GE Healthcare Dharmacon Inc.) and ON-TARGET plus Non-targeting Pool siRNA (cat no. D-001810-10-05, GE Healthcare Dharmacon Inc.). The required amount of SMART-pool target siRNA (Dharmacon) and 5 μL of Lipofectamine RNAiMAX (Invitrogen) were each diluted into a final volume of 250 μL in Opti-MEM (GIBCO), combined, gently mixed, and incubated at room temperature for 20 min. 500 μL of this transfection solution was overlaid onto cells at a final concentration of 80 nM siRNA. Transfection of SK-N-MC cells with ON-TARGET plus Non-targeting Pool (Dharmacon, with no significant homology to any known gene sequences from mouse, rat, or human) served as a negative control. Cells were treated with clenbuterol (10 μM) 24 hours after transfection and lysed after 48 hours of treatment by TRIzol reagent for mRNA and 1× PBS+0.5% NP-40 for protein extraction.

### RNA Extraction and qPCR

Total RNA from flash-frozen mice brain tissue, rodent primary cells, human neuroblastoma cells and iPSC-derived neuronal cells was isolated using TRIzol® (Life Technologies) following the manufacturer's protocol. RNA concentration was measured using NanoDrop spectrophotometer (Thermo Fisher Scientific, Wilmington, De.). 2 μg of total RNA was reverse transcribed into cDNA using the High Capacity cDNA Reverse Transcription Kit from Applied Biosystems. Quantitative PCR was performed using TaqMan Universal PCR Master Mix (Applied Biosystems) on an ABI 7900HT instrument (Applied Biosystems) as described previously (31). For mouse brain tissue and rat primary cortical neuron samples, rodent-specific TaqMan Assay on-demand primers and probes were used to assay the relative abundance of target genes, and rodent Rp113 was used as a reference gene to normalize for RNA loading. Similar results were obtained when Actb or Ubc were used as controls for input RNA. For human neuroblastoma and iPSC-derived neuron samples, human TaqMan Assay on-demand probes were used to assay the relative abundance of target genes, and the geometric mean of the three human reference genes RPL13, GUSB and UBC was used to normalize for RNA loading. Expression values were analyzed using the comparative threshold cycle method (31).

### Protein Extraction

Protein lysis buffer containing 320 mM sucrose, 5 mM NaF, 1 mM Na₃VO₄, 10 mM Tris (pH 7.4), 1 mM EGTA, and 1 mM EDTA was used for mice brain tissue lysis. 100 μL of cold extraction buffer was added to each sample and homogenized in a dounce homogenizer. After incubating on ice for 10 minutes post-homogenization, samples were centrifuged at 10,000 rpm at 4° C. for 10 minutes. Supernatant was transferred to new tubes and centrifuged again at 10,000 rpm at 4° C. for additional 10 minutes. The final supernatant was transferred to new tubes and protein estimation was performed by ELISA. Total protein from rodent primary cells, human neuroblastoma cells and iPSC-derived neuronal cells was extracted using lysis buffer containing 1×PBS and 0.5% NP-40. Cells were washed once with cold PBS and lysed by adding cold lysis buffer. The cells were collected by scraping, then incubated for 30 minutes by rotating at 4° C. The samples were centrifuged at 14,000 rpm at 4° C. for 10 minutes and supernatant was transferred to new tubes. Total protein concentrations were determined by BCA assay (Thermo Scientific) according to the manufacturer's instructions. Total α-synuclein was quantified using an α-synuclein-specific sandwich ELISA.

### Antibodies

Antibodies used were anti-α-synuclein, clone 2F12 (11) (EMD Millipore, 1:1000 for Western blotting of endogenous α-synuclein, also used for ELISA), anti-α-synuclein, clone SOY1 (11) (EMD Millipore, used for ELISA), monoclonal anti-α-Synuclein Syn-1 (Clone 42, Becton-Dickinson; used for ELISA), anti-α-synuclein, clone 1H9 (courtesy Dr. Selkoe lab; used for ELISA), anti-α-synuclein, C-20 (Santa Cruz Biotechnology, 1:1000 used for Western blotting), anti-Histone 3 (acetyl K27) (abcam, 1:1000 for Western blotting, also used for ChIP assay) and anti-α-tubulin (Cell Signaling Technology, 1:1000 for western blotting).

### α-Synuclein-Specific ELISAs

An α-synuclein-specific Meso Scale Discovery (MSD) ELISA assay (described in Ref. (11)) was utilized for endogenous α-synuclein protein measurement. 96-well Multi-Array Standard Bind plates (MSD, Rockville, Md.) were coated with the capture antibody 2F12 (11) diluted to 6.7 ng/μl in phosphate-buffered saline in 30 μvolumes/well and incubated at 4° C. overnight. Following emptying of the wells, plates were blocked for 1 hour at room temperature in blocking buffer (5% MSD Blocker A; TBS-T). After 3 washes with TBS-T, samples diluted in TBS-T with 1% MSD Blocker A and 0.5% NP-40 were loaded and incubated at 4° C. overnight. After samples were emptied and plate washed 3 times with TBS-T, sulfo-tagged SOY1 mAb (detection Ab) (generated using Sulfo-Tag-NHS-Ester (MSD) diluted in blocking buffer (6.7 ng/μl), was added to the plate (30 μl volumes/well) and shaken for 1 hour at room temperature. Following 3 washes, MSD Read (2×, diluted in ultrapure water) buffer was added and the plates were immediately measured using a MSD Sector 2400 imager. For confirmatory studies, an second α-synuclein-specific ELISA implementation was used employing a different, extensively published anti-α-synuclein detection antibody (Syn-1 mAb) and a different anti-α-synuclein capture antibody (1H9, courtesy Dr. Selkoe).

### Immunoblotting

For western blot analysis, protein samples (20 μg) were run on a 4-20% gradient SDS-polyacrylamide gel (Biorad) and transferred onto a polyvinylidene fluoride (PVDF) membrane filter (0.2 μm; Biorad) using an electroblot apparatus (Bio-Rad, Hercules, Calif.) at 100V for 1 h in transfer buffer (25 mM Tris-HCl, 192 mM glycine, 0.1% SDS, 20% (v/v) methanol). The membrane was incubated in blocking solution (50 mM Tris-HCl, 200 mM NaCl, 1 mM MgCl₂, pH 7.4) containing 5% non-fat dry milk powder for 1 hour at room temperature. The membrane was processed through sequential incubations with primary antibody overnight at 4° C. followed by incubation with horseradish peroxidase-conjugated species specific secondary antibody at 1:250 dilutions (Thermo Scientific Pierce). Immunoreactive proteins on the membrane were visualized using the SuperSignal™ West Pico Chemiluminescent kit (Thermo Scientific Pierce). Scanned western blots were analyzed using ImageJ software, version 1.47. Pictures were inverted and usually the background signal from an empty lane was subtracted to obtain specific signals for each lane.

### Chromatin Immunoprecipitation Assay

Chromatin immunoprecipitation (ChIP) assays were performed using the Simple ChIP Enzymatic Chromatin IP kit protocol as recommended by the manufacturer (Cell Signaling Technology) with slight modifications. Briefly, ChIPs were performed on asynchronously growing SK-N-MC cells treated with clenbuterol (10 μM), valproic acid (100 μM) or propranolol (100 μM). Cross-linking was carried out with 1% formaldehyde for 10 min at room temperature. Cross-linking was subsequently quenched by adding glycine to a final concentration of 1× for 5 min. Cells were collected and washed twice with PBS, then resuspended in 2 mL of lysis buffer (as described in the protocol). After 10 min on ice, cells were treated with micrococcal nuclease and then sonicated to obtain DNA fragments of ˜500 bp as determined by agarose gel electrophoresis with ethidium bromide staining. Protein-DNA complexes were isolated by centrifugation at 10,000 rpm for 10 min. Supernatants with protein-DNA complexes were incubated overnight with rabbit polyclonal antibody directed against acetylated H3K27 (Abcam). Normal rabbit IgG was used as a negative control and Histone H3 rabbit mAb (ChIP formulated) as positive control. 2% of sample was saved as input. Antibody-protein-DNA complexes were further incubated for 2 hours with 30 μl of ChIP-grade Protein G magnetic beads to isolate antibody bound fractions of chromatin. Immuno-complexes were washed with the buffers provided and as described in the kit. Protein-DNA complexes were eluted and cross-links of pull down fractions and inputs (2% of total IP fraction) were reversed by 2 hours incubation in Proteinase K and 5 M NaCl at 65° C. DNA was then extracted, purified, precipitated, and resuspended in TE for qPCR. 2 μl of immunoprecipitated DNA was analyzed by qPCR using a Power SYBR Green PCR Master mix (Applied Biosystems by Life technologies) on an ABI 7900HT instrument (Applied Biosystems) with the following temperature profile: 3 min of enzyme activation at 95° C. followed by 40 cycles of 95° C. for 15 s and 60° C. for 60 s. Primers used for qPCR are listed in Table 2. Sample from three independent immunoprecipitation assays were analyzed.

TABLE 2
Primers used for qPCR

| CHIP assay | Forward Primer | Reverse Primer |
|---|---|---|
| Promoter | ACTTAACGTGAGGCGCAAAA | CTATCTCGGATGGGGATGG |
| (insert 1) | (SEQ ID NO: 5) | (SEQ ID NO: 6) |
| Enhancer | GACAAGGGAAGGTGGATGAA | GGGGATGCTTTTACCAGTGA |
| (insert 2) | (SEQ ID NO: 7) | (SEQ ID NO: 8) |
| Enhancer | CACAATTGGCCCTGGATTAG | TCCCTGTTTCCTCTTTGTGG |
| (insert 3) | (SEQ ID NO: 9) | (SEQ ID NO: 10) |

### Mitochondrial Superoxide and Viability Assay for iPSC-Derived Neuronal Precursor Cells (NPC)

Patient-derived NPCs carrying the familial SNCA triplication were generated as described previously (28). NPCs were seeded at 3.5×10⁴ cells/mm² in 96-well plates (Greiner Cellcoat μClear) for HTS plate reader analysis (Polarstar Omega, BMG). For experiments with toxicants and inhibitors, NPCs were cultured under the following conditions for 18 hours unless specified differently in the experiment procedures: Standard NPC growth medium, growth medium plus 20 μM rotenone (Sigma).

For the Plate Reader HTS, each experiment was conducted with 6 replicates per cell line and treatment regimen. NPCs were treated with 20 μM clenbuterol for four days. Signals from individual wells were acquired with integration of fluorescence signals over 1 second duration by bottom read in orbital mode (8-spot measurements with 2 mm radius). Data analysis was performed using BMG Mars software. For data analysis of individual experiments, the mean±SD was plotted using GraphPad7. Non-directional Student's t-test was used for direct statistical comparisons. For multiple comparisons, 2-way and multivariant ANOVA were used. Where significant F-values were obtained, pairwise comparisons were made using Tukey's post hoc analysis. Differences were considered statistically significant at p≤0.05.

For the MitoSox assay for the detection of mitochondria-associated superoxide levels, adherent NPCs in 96-well plates were incubated with 2 μM MitoSOX™ (Ex./Em. 510/580 nm) and MitoTracker® Green (485/520 nm) (both Life Technologies) in high glucose without phenol red (Life Technologies), containing supplements without antioxidants (Life Technologies) for 15 min at 37° C. in the dark. Cells were then washed twice with medium (also containing 1 μM Hoechst 33342). Fluorescence was detected by sequential readings, and MitoSOX™ signals were normalized to mitochondrial content (Mitotracker®) and cell number (Hoechst).

For the Resazurin assay for reduction potential by NADPH levels, NPCs were prepared as above and then loaded with 3 μM C12-Resazurin (Ex/Em: 563 nm/587 nm) (Life Technologies) in medium (see above), for 30 min at 37° C. Loading solution was then removed and cells were washed twice with full growth medium containing 1 μM Hoechst 33342. Relative C12-Resazurin fluorescence intensities were normalized to Hoechst 33342 fluorescence.

### Statistical Analyses of SNCA Expression

Two-tailed Student's t-tests were used for two-group comparisons as indicated in the figure legends; one-way or two-way ANOVAs with Tukey's post-hoc test were used for analyses involving multiple groups as indicated in the figure legends. P-values equal to or lower than 0.05 were considered statistically significant.

### Analyses Using the Norwegian Prescription Database

We included the entire population of Norway alive on 1 Jan. 2004 by linking the Norwegian National Registry (NNR) with the Norwegian Prescription Database (NorPD) using the unique national identification number. The NNR contains information on all residents in Norway, time of immigration, emigration and death. This information was further linked to the Norwegian National Education Database (NNED) to assess level of education of all participants.

### NorPD

This registry contains complete information on all prescribed drugs dispensed at pharmacies to individuals since 2004. Each prescription includes the national identification number, which makes it possible to generate a precise chronological prescription record for individuals over time, and to link this information to other registries. Drugs are classified according to the Anatomical Therapeutic Chemical classification and doses are reported as defined daily dose (DDD). If the national insurance scheme covers the cost of the drug, the prescription also contains the diagnosis the drug was prescribed for (from 2008: ICD-10 and ICPC; before 2008: local diagnostic code).

### Definition of Outcome

We defined an incident case of PD as an individual who had received at least 365 DDD of levodopa (NO4BA), which corresponds to one year of use at the average maintenance dose per day of the drug, and who was given a diagnosis of PD (ICD-10: G20) as the reason for the prescription. The first year of prescription of any antiparkinson drug (N04) was considered year of onset of PD. We used the year 2004 as a washout-period to only include incident cases. By using this definition, we found a total of 10,070 individuals with PD, of whom 4,634 were incident cases in the period 2005-2014. This gave a mean annual incidence rate of 10.3 per 100,000, which is very similar to what was reported in the most recent large clinical epidemiological study of PD in Norway (12.6 per 100,000) (17). The mean age of first prescription of antiparkinson drug was 71.3 years with 58.3% males compared to 69.4 years at onset and 57% males in the clinical study.

### Exposure Definition

There were 619,863 users of salbutamol (ATC: R03AC02 (inhalable) and R03CC02 (systemic)) and 273,356 users of inhaled corticosteroids (ATC: R03BA) during follow-up, while there were 295,387 users of salbutamol (ATC: R03AC02 (inhalable) and R03CC02 (systemic) during 2004-2007. Participants were categorized as exposed at time of first prescription of the relevant drug. In the same period, there were 63,210 users of propranolol (ATC: C07AA05), of whom 33,864 were prescribed the drug because of non-neurological indications covered by the national insurance scheme. Specifically, this included the ICD-10 codes −22, G43, I10, Ill, 112, 113, 115, 120, 121, 122, 147, 148, 149 and Z94, the ICPC codes—22, −51, K74, K75, K78, K79, K80, K86, K87 and K89, and the local diagnostic code 012 (Morbus Cardiovascularis). To further limit the chance of reverse causality, we only included users who had been prescribed at least 365 DDD or more of propranolol. This included 14,794 users and these individuals were categorized as exposed at time of prescription of propranolol where 365 DDD was exceeded. For the period 2004-2007, there were 9,339 users of propranolol fulfilling the same criteria.

### Statistical Analysis

The level of education was categorized into primary education, secondary education, undergraduate education or graduate education. Age at study start was categorized in 5 years intervals. We calculated risk estimates (hazard ratio) with 95% confidence intervals using a Cox proportional hazard regression model, which included the exposure of interest (salbutamol, propranolol or corticosteroids) as a time-dependent covariate adjusting for sex, age and level of education. Outcome was PD as defined above at time of first prescription of an antiparkinson drug. Participants not developing PD were censored at end of follow-up (31 Dec. 2014), at date of death or date of emigration out of Norway, whichever occurred first. To test for dose-response effects, we categorized participants according to the cumulative number of DDD in 2004-2007 and estimated the RR of developing PD in 2008-2014. Those who developed PD, died or moved out of Norway before 2008 were excluded from these analyses (n=189,584). The hazard ratios were interpreted as rate ratios.

Information on data access to NorPD is available on the world wide web at norpd.no; skatteetaten.no/en/person/National-Registry/This-is-the-National-Registry/; and ssb.no/en/omssb/tjenester-og-verktoy/data-til-forskning/utdanning.

### Post-Mortem Brain CAGE Sample Preparation and RNA Extraction

Postmortem substantia nigra tissue samples were obtained from University of Maryland, University of Washington, and McLean Hospital (Belmont, Mass.). Four human post-mortem samples (healthy controls) were utilized for CAGE. 5 μg of total RNA was exacted from each sample using the RNeasy RNA Kit (Qiagen) with an RNA integrity number (RIN)>6. Postmortem brains were collected under local IRB approval. Use of postmortem samples for expression analysis was approved by the IRB of Brigham & Women's Hospital.

### Library Preparation

Libraries were constructed using a published CAGEseq protocol adapted for next-generation sequencing (37). Briefly, complementary DNA (cDNA) was synthesized from total RNA using random primers, and this process was carried out at high temperature in the presence of trehalose and sorbitol to extend cDNA synthesis through GC-rich regions in 5′ untranslated regions. The 5′ ends of messenger RNA within RNA-DNA hybrids were selected by the cap-trapper method and ligated to a linker so that an EcoP15I recognition site was placed adjacent to the start of the cDNA, corresponding to the 5′ end of the original messenger RNA. This linker was used to prime second-strand cDNA synthesis. Subsequent EcoP15I digestion released the 27-base pair (bp) CAGEseq reads. After ligation of a second linker, CAGEseq tags were polymerase chain reaction amplified, purified, and sequenced on the HiSeq 2000 (Illumina) using standard protocol for 50 bp single end runs.

### CAGE Expression Analysis

CAGEseq data were filtered for CAGEseq artifacts using TagDust (version 1.12), removal of reads mapping to known ribosomal RNA genes and low quality reads, mapping to the human genome (hg19) using Burrows-Wheeler Aligner (version 0.5.9) for short reads. Reads mapping to autosomes were used to minimize gender and normalization biases for subsequent analysis. Normalization was done based on the amount of reads per million sequence reads.

### Example 1. B2-adrenoreceptor is a Regulator of the α-synuclein Gene

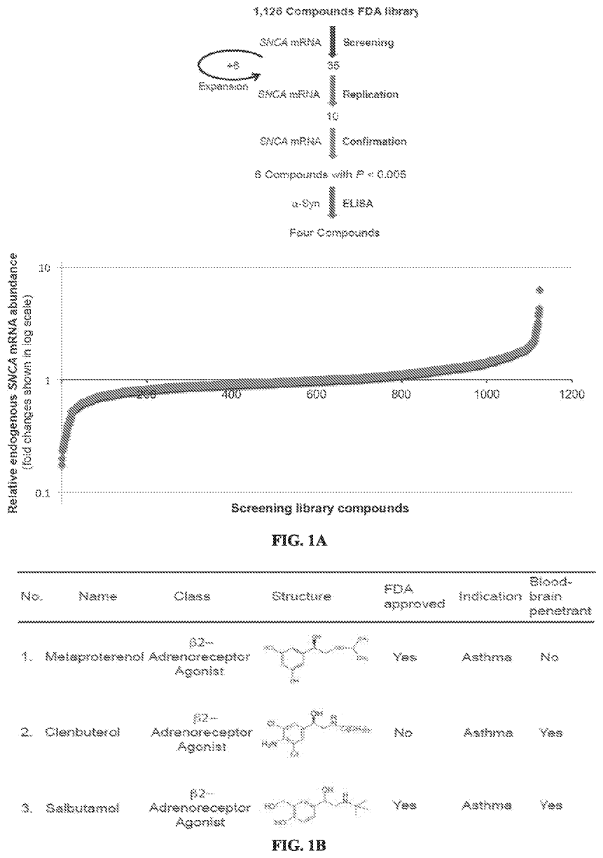

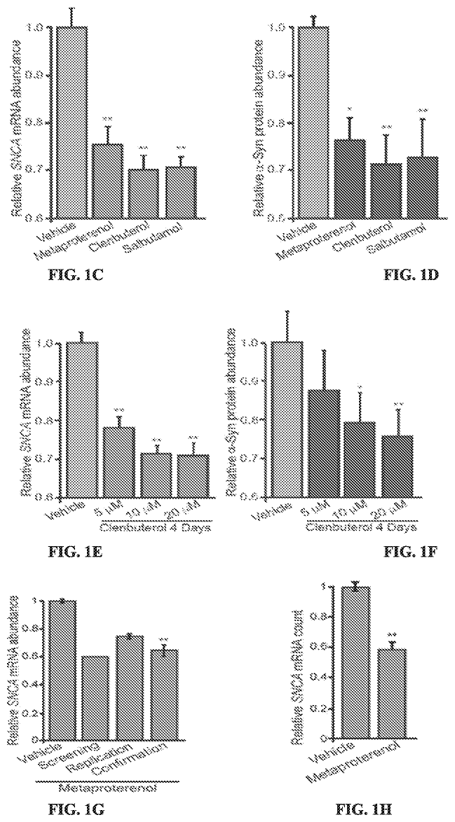

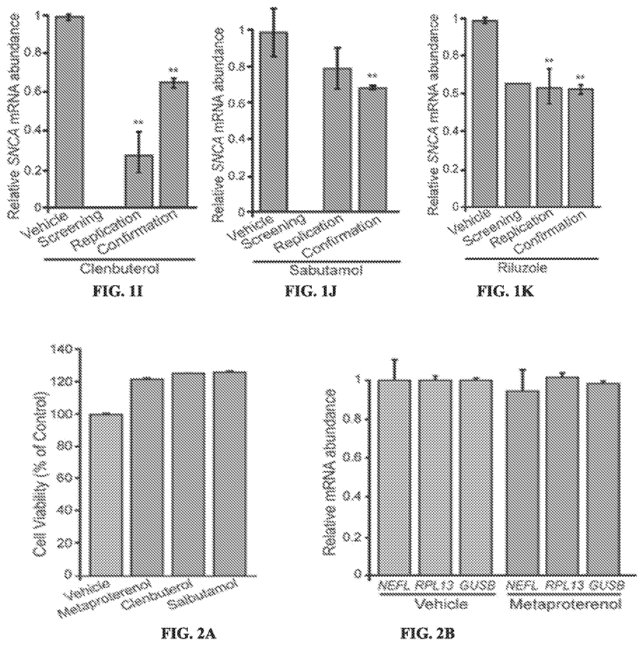

SNCA expression-lowering compounds were identified in a four-stage study design (FIG. 1A) consisting of screening, replication, and confirmation of transcript expression, followed by an ELISA stage for protein expression quantification. 1,126 FDA-approved drugs and a diverse set of natural products, vitamins, health supplements, and alkaloids (“FDA library”) were screened. Human neuroblastoma SK-N-MC cells were treated with each compound for 48 hours. Forty-one compounds were included in the replication stage: 35 compounds lowered SNCA expression by more than 35% in the screening stage (including the selective β2-adrenoreceptor (AR) agonist metaproterenol). Six related drugs were added at the replication stage (“hit expansion”; including the selective β2AR agonists, clenbuterol and salbutamol). Four compounds achieved P values ≤0.005 by two-tailed Student's t-test in the confirmation stage (two-tailed Student's t-test) and also lowered α-synuclein protein abundance by ELISA in SK-N-MC cells compared to vehicle with P≤0.05 (FIG. 1A; two-tailed Student's t-test). Surprisingly, three of these hits were β2-adrenoreceptor (AR) agonists (FIG. 1B) and prioritized for further investigation.

Treatment with metaproterenol reduced SNCA mRNA abundance in SK-N-MC cells compared to control cells (P=0.005, two-tailed Student's t-test) in the confirmation stage (FIG. 1G) and was further verified (FIG. 1H). Treatment with clenbuterol (FIG. 1I) and salbutamol (FIG. 1J) also exhibited similar effects on relative SNCA mRNA abundance. Thus, β2AR activation can regulate endogenous SNCA expression in SK-N-MC cells. Interestingly, the screen highlighted riluzole hydrochloride (FIG. 1K) as fourth hit. It is FDA-approved for modification of amyotrophic lateral sclerosis and attenuates dopaminergic neurodegeneration in a 6-hydroxydopamine rat model of PD (9).

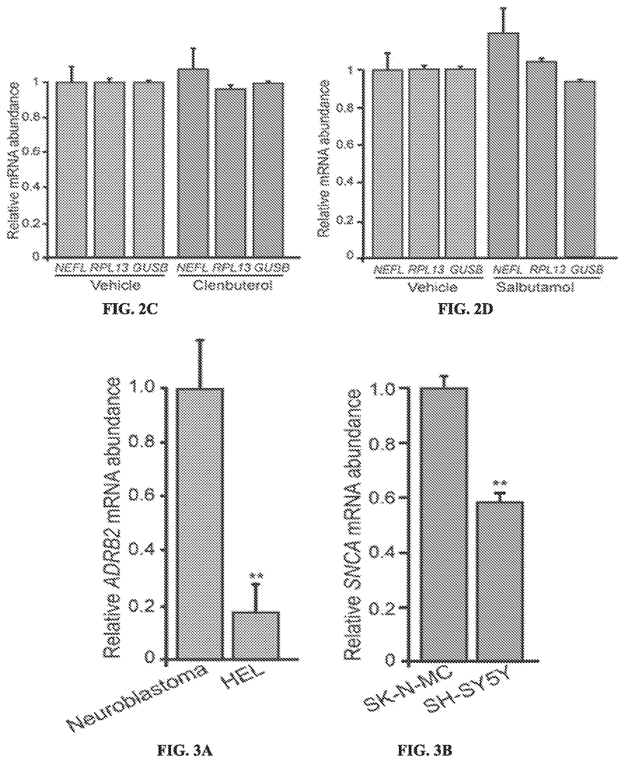

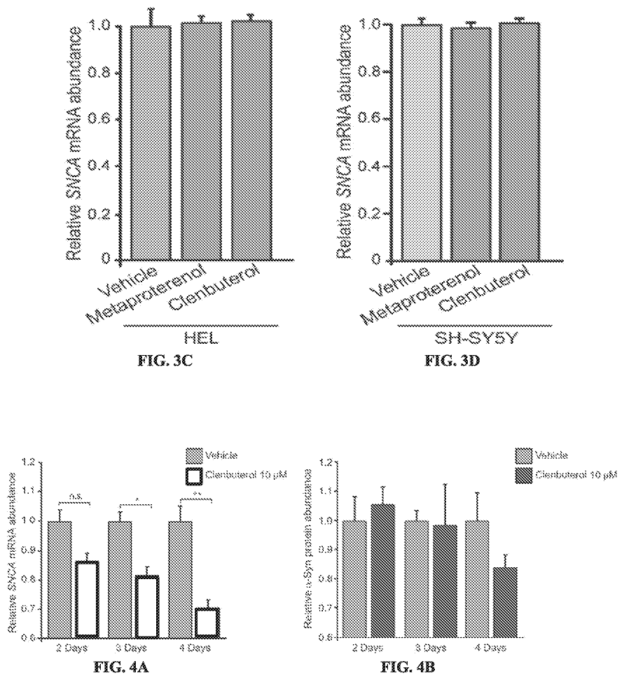

β2AR activation selectively modulated the expression of SNCA without adversely affecting neuronal cell viability or housekeeping gene expression (FIGS. 2A-D; 10). As expected, the effects of β2AR agonists on SNCA expression were dependent on cellular context (FIGS. 3A-D). For example, in human erythroleukemia (HEL) cells, which express SNCA mRNA but lack β2AR (FIG. 3A) or in neuronal SH-SYSY cells, which transcribe β2AR, but express low levels of SNCA mRNA (FIG. 3B), agonists did not influence SNCA expression (FIGS. 3C-D).

A sensitive ELISA and anti-α-synuclein antibodies (11) were used to determine whether the modulation of SNCA mRNA expression via β2AR translates into changes in α-synuclein protein abundance. In rat primary cortical neurons endogenous Snca mRNA (FIG. 1C) and α-synuclein protein levels (FIG. 1D) were significantly, but modestly, reduced, in response to β2AR activation by metaproterenol (<0.005 and <0.05), clenbuterol (P<0.005) or salbutamol (P<0.005) compared to controls by ANOVA with Tukey's.

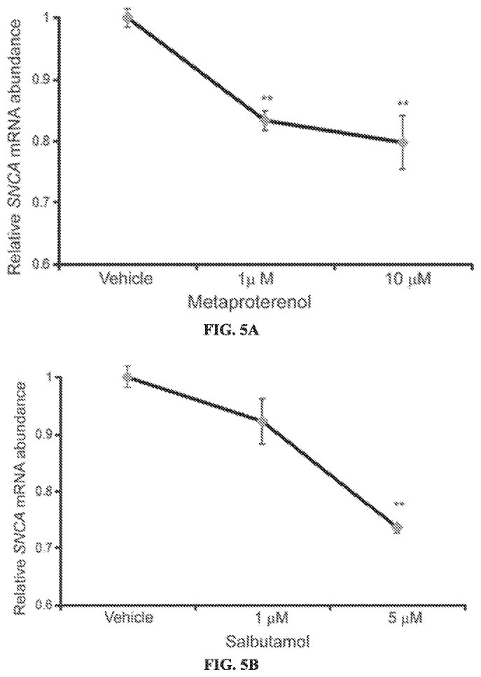

β2AR agonists lowered SNCA expression in a dose- and time-dependent manner (10; FIGS. 4A-B). Increasing concentrations of clenbuterol (5, 10, 20 μM) correlated with a decrease in α-synuclein mRNA (FIG. 1E) and protein (FIG. 1F) levels in SK-N-MC cells. Similarly, metaproterenol and salbutamol lowered SNCA mRNA expression in a dose-dependent manner (P values <0.005, ANOVA with Tukey's; FIGS. 5A-B).

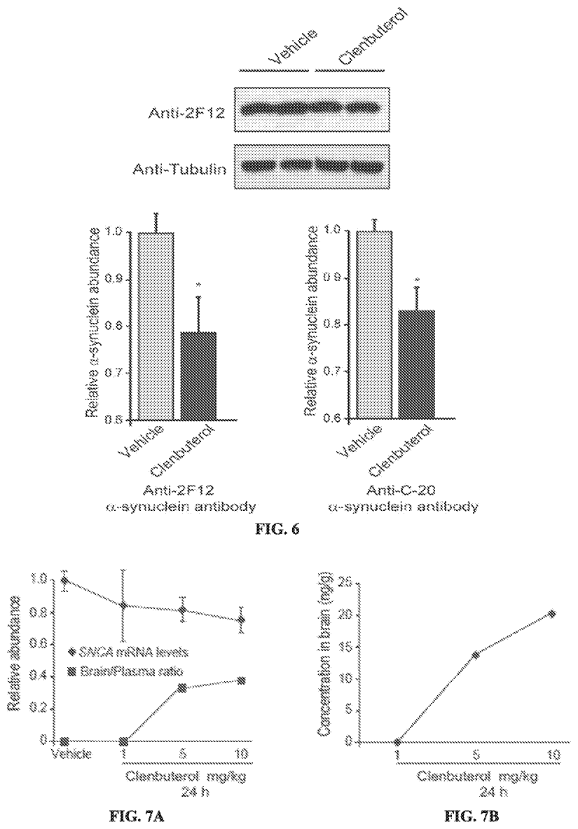

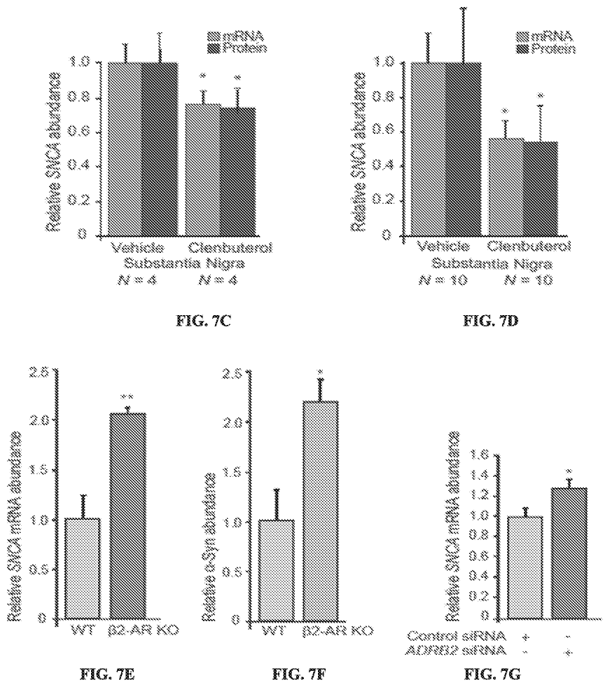

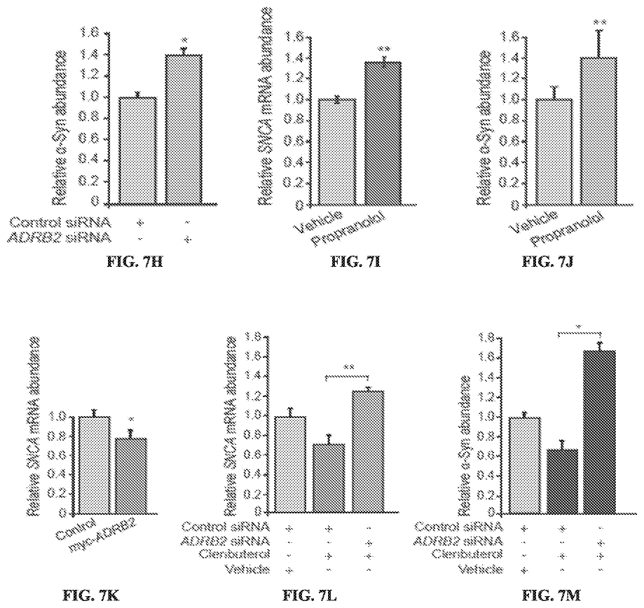

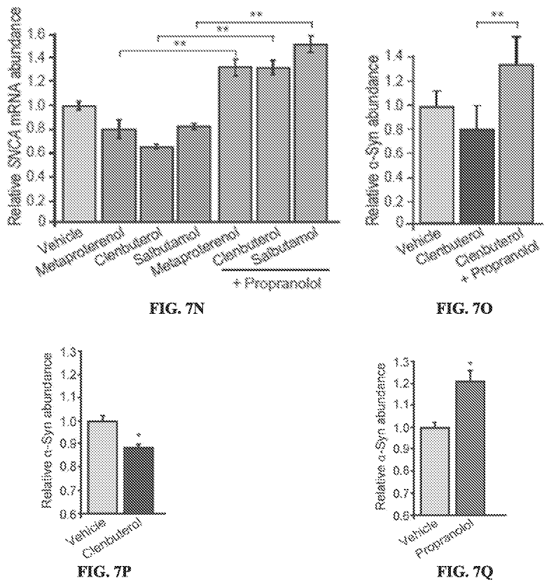

PD preferentially affects dopaminergic neurons in the substantia nigra. The effects of the selective β2AR agonist clenbuterol (which is administered efficiently intraperitoneally) were examined to probe the effects of β2AR activation on Snca expression in the substantia nigra of wild type C57BL/6J mice. As expected (12, 13), clenbuterol crossed the blood-brain barrier and its brain/plasma ratio increased with doses of 1, 5, or 10 mg of drug per kilogram body weight compared to controls (FIG. 7A).

Intraperitoneal injection of 10 mg/kg body weight administered for 24 hours achieved the highest brain/plasma ratio (FIG. 7A) and brain concentration (FIG. 7B) and induced a significant reduction in nigral α-synuclein protein and mRNA levels (P<0.05, respectively, two-tailed Student's t-test) (FIG. 7C). Next, a larger, randomized, blinded, placebo-controlled trial was performed in mice to determine whether clenbuterol is efficacious in lowering α-synuclein expression in the substantia nigra of wild-type mice. Mice were euthanized after 24 hours of acute drug treatment. β2AR activation lowered the expression of endogenous α-synuclein protein and mRNA levels in the PD-vulnerable substantia nigra, and SK-N-MC cells after 14 days (P=0.01, respectively, two-tailed t-test) (FIG. 7D and FIG. 12). This was confirmed by Western blotting with various anti-α-synuclein antibodies (FIG. 6). Overall, β2AR agonist treatment reduced SNCA expression by about 20-30% in rodent neurons and substantia nigra.

Snca expression levels were examined in primary neurons derived from mice carrying a deletion of the β2AR gene (Adrb2). Endogenous Snca mRNA and protein levels were increased by 100% and 120% compared to controls (P values 0.004 and 0.01, respectively, t-test) (FIGS. 7E and F). In accord, silencing of β2AR in human SK-N-MC cells increased α-synuclein mRNA and protein levels (FIGS. 7G and H).

Moreover, chemical antagonism of the β2AR with propranolol, a well characterized β blocker, in SK-N-MC cells similarly increased endogenous SNCA mRNA and protein (P values of 0.00001 and 0.001, respectively, two-tailed Student's t-test) (FIGS. 7I and J). Conversely, transient transfection of SK-N-MC cells with ADRB2 constructs reduced endogenous SNCA mRNA levels compared to controls (P=0.01) (FIG. 7K).

Taken together, this suggests that β2AR-modulation is sufficient for altering endogenous SNCA expression. Genetic silencing of the β2AR or co-treatment with propranolol each blocked clenbuterol's SNCA expression-lowering effects (FIGS. 7L-Q). Collectively, these internally consistent data suggest that β2AR modulation is sufficient for altering SNCA expression and necessary for mediating the effects of β2AR ligands on endogenous SNCA expression.

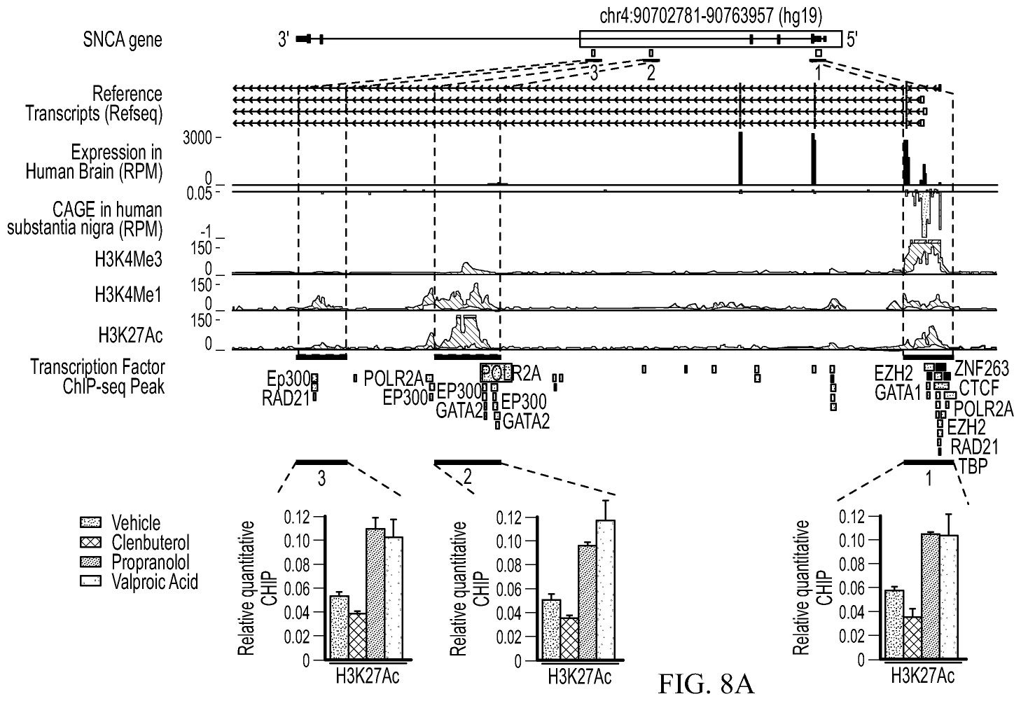

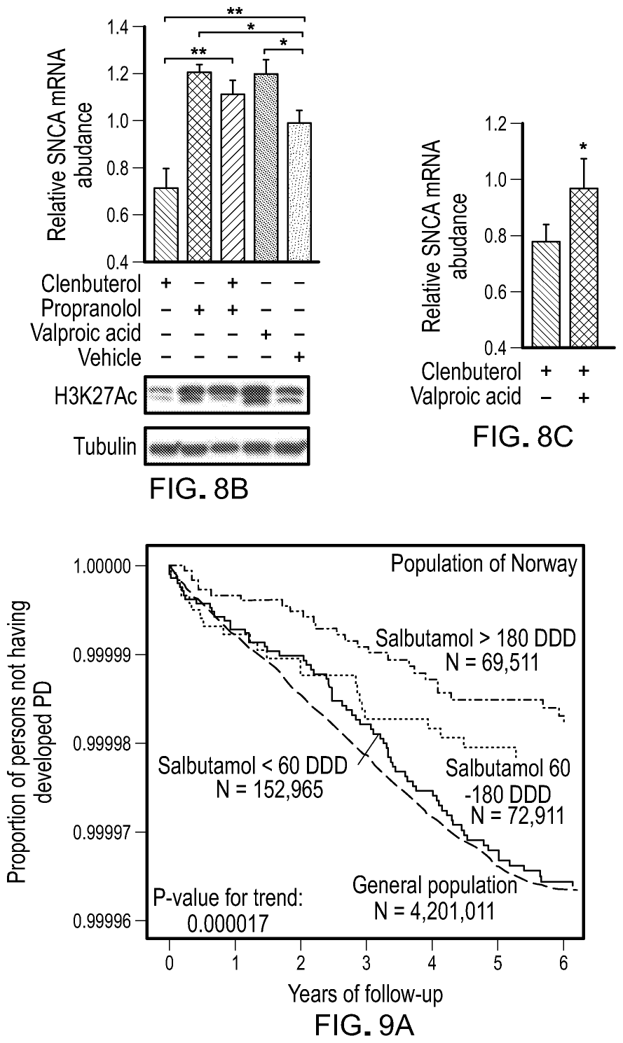

SNCA transcription appears finely regulated through a classical promoter spanning the non-protein-coding exon 1 and intron 1 at the 5′ end of the SNCA locus and enhancers in the long intron 4 (FIG. 8A; 5). The endogenous SNCA promoter and putative enhancer sites were clarified using Cap analysis gene expression (CAGE) in human PD-relevant substantia nigra, and by integrative genomics (FIG. 8A; 10). H3K27ac signals (indicative of active enhancer elements) were observed at the promoter and the enhancer regions (FIG. 8A).

Since β2AR-stimulation has been implicated in regulating WNK4 transcription via histone acetylation in renal cells (14), it was determined whether β2AR activation can regulate SNCA transcription through an analogous mechanism. Clenbuterol reduced H3K27 acetylation precisely across promoter (FIG. 8A; site 1) and two putative intronic enhancers (FIG. 8A; sites 2, 3) compared to vehicle with P<0.05 by one-way ANOVA with Tukey's. Conversely, the β blocker propranolol increased H3K27Ac across these putative regulatory sites (FIG. 8A; P<0.05). Consistently, the known histone deacetylase inhibitor, valproic acid (15), increased H3K27 acetylation (FIG. 8A). Western blotting with an antibody against H3K27Ac confirmed our hypothesis (FIG. 8B). Clenbuterol treatment resulted in a correlated decreased in H3K27Ac levels and relative SNCA mRNA abundance (FIG. 8B). Conversely, treatment with valproic acid resulted in an increase in H3K27Ac levels and in relative SNCA mRNA abundance compared to vehicle-treated cells (FIG. 8B). Inhibition of H3K27 deacetylation (by co-treatment with valproic acid) abrogated the β2AR agonist effect on SNCA expression (FIG. 8C). Thus, β2AR regulates the transcription of α-synuclein in correlation with H3K27 acetylation across promoter and enhancers in the human SNCA locus.

The effects of β2AR activation were evaluated on disease modification in two nation-wide, longitudinal analyses of incident PD in Norway, a mouse model of MPTP-induced human parkinsonism, and in an iPSC-derived neuronal culture system from a patient with autosomal dominant PD due to a triplication of the SNCA locus. The Norwegian Prescription Database (NorPD) contains complete information on all prescribed drugs dispensed at pharmacies to individuals in Norway since 2004 (16).

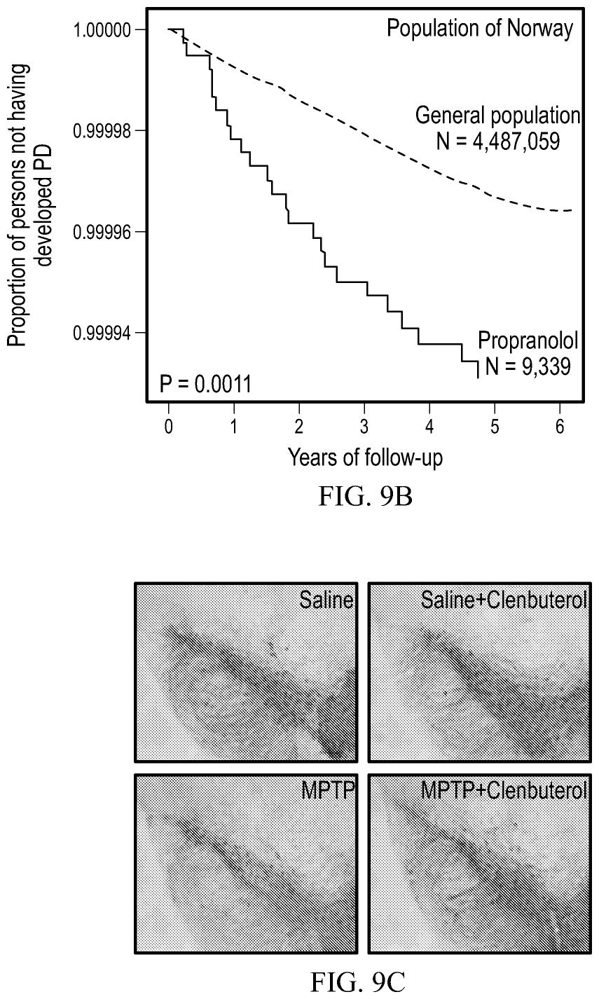

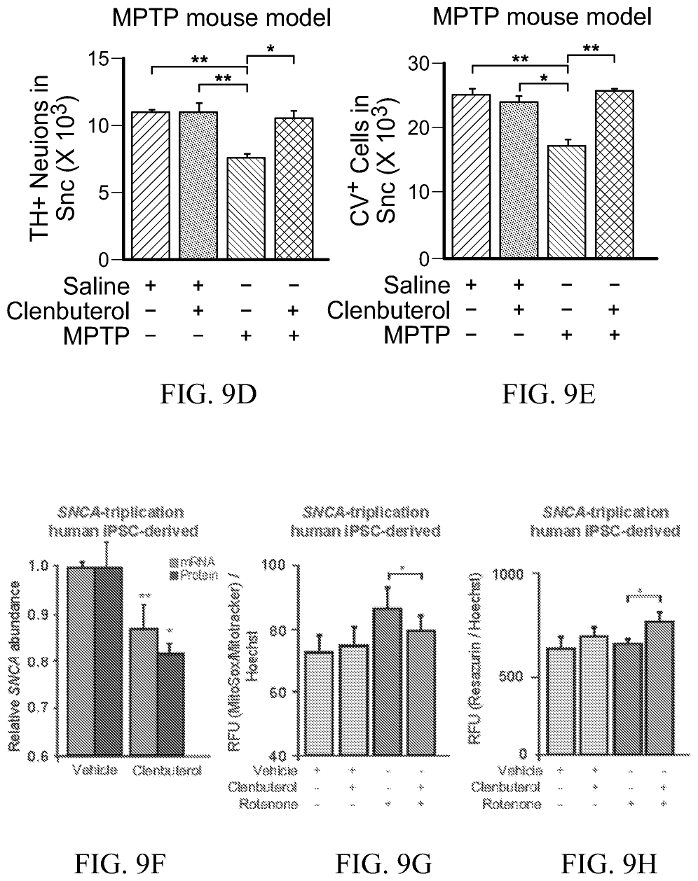

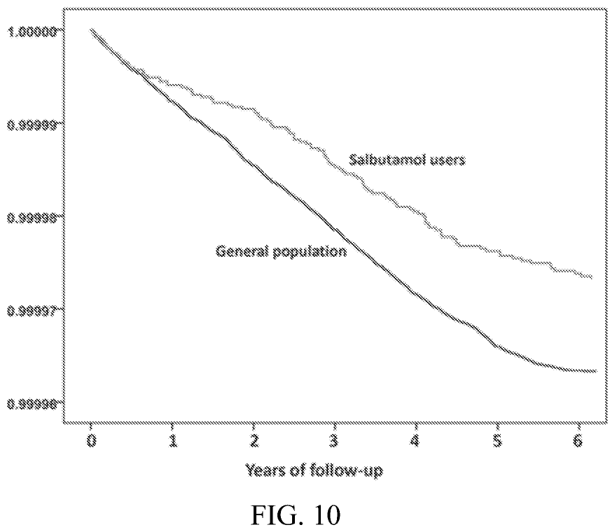

As β2AR modulates SNCA expression, it was determined whether use of β2AR-ligands would affect PD risk. Salbutamol and propranolol, the most commonly used β2AR-agonist and β2AR-antagonist in Norway were tested as time-dependent covariates in two separate Cox proportional hazard models adjusting for sex, age and level of education, including the total Norwegian population alive on Jan. 1, 2004 as study population (N=4.6 millions). A yearly incidence rate similar to a recent clinical incidence study in Norway was observed (10, 17). Salbutamol was associated with decreased risk for PD with a rate ratio (RR) of 0.66 (95% confidence interval (CI), 0.58-0.76; Tables 3, 4; FIG. 9A, FIG. 10).

TABLE 3
Rate ratio (RR) for Parkinson's disease in persons treated
with salbutamol or propranolol during a complete
11 year follow-up of the entire population of Norway

| | | | | RR (95% CI) | |
|---|---|---|---|---|---|
| | | | Person- | Age-, sex- | Multivariate |
| | Users | Cases | years | adjusted | adjusted^{a} |
| Salbutamol | | | | | |
| Never user | 4,066,119 | 4,398 | 36,700,554 | 1 (ref) | 1 (ref) |
| Ever user | 619,863 | 236 | 3,135,956 | 0.65 | 0.66 |
| | | | | (0.57-0.74) | (0.58-0.76) |
| Propranolol | | | | | |
| Never user | 4,671,188 | 4,593 | 39,770,912 | 1 (ref) | 1 (ref) |
| Ever user | 14,794 | 41 | 65,598 | 2.16 | 2.20 |
| | | | | (1.59-2.94) | (1.62-3.00) |

^{a}Adjusted for age in 5 year periods, sex and level of education.
^{b}Use of at least 365 defined daily doses
RR: Rate ratio; PD: Parkinson's disease; CI: confidence interval

TABLE 4
Rate ratio (RR) for Parkinson's disease during
2008-2014 for salbutamol prescribed during
2004-2007 in the entire population of Norway

| | | | RR (95% CI) |
|---|---|---|---|
| | Users | Cases | Multivariate |
| | 2004-07 | 2008-14 | Adjusted^{b} |
| Salbutamol | | | |
| Never user | 4,201,011 | 2,338 | 1 (ref) |
| Low (<60 DDD) | 152,965 | 68 | 0.96 (0.76-1.23) |
| Medium (60-180 DDD) | 72,911 | 23 | 0.60 (0.40-0.91) |
| High (≥180 DDD) | 69,511 | 25 | 0.45 (0.31-0.67) |

^{a}Adjusted for age in five year periods, sex and level of education.
DDD: Defined daily dose; RR: Rate ratio; CI: confidence interval

Propranolol was associated with markedly increased risk of PD with a RR 2.20 (95% CI 1.62- 3.00; Table 5, FIG. 9B). The most common indication for salbutamol in the database was asthma. Smoking has been associated with decreased risk for PD (18). Tobacco exposure, is also associated with early childhood asthma (19). However, inhaled corticosteroids, frequently prescribed for asthma, did not reduce the PD risk (RR 0.95, 95% CI 0.80-1.12; Table 5) after adjusting for salbutamol use and level of education. Further, adjusting for education, which is strongly associated with smoking habits in Norway (20), a slight change in the effect of salbutamol was observed (Table 5). Thus, it is unlikely that smoking can fully explain the association between salbutamol and PD.

TABLE 5
Rate ratio for Parkinson's disease according to use
of salbutamol and corticosteroids in a complete
11 years follow-up of the total Norwegian population.

| | | | | RR (95% CI) | |
|---|---|---|---|---|---|
| | | | | Multi- | Multi- |
| | | | Person- | variate | variate |
| | Users | Cases | years | adjusted^{a} | adjusted^{b} |
| Salbutamol | | | | | |
| Never user | 4,066,119 | 4,398 | 36,700,554 | 1 (ref) | 1 (ref) |
| Ever user | 619,863 | 236 | 3,135,956 | 0.66 | 0.67 |
| | | | | (0.58-0.76) | (0.59-0.77) |
| Corticosteroids | | | | | |
| Never user | 4,412,626 | 4,480 | 38,262,872 | 1 (ref) | 1 (ref) |
| Ever user | 273,356 | 154 | 1,573,638 | 0.84 | 0.95 |
| | | | | (0.71-0.98) | (0.80-1.12) |

^{a}Adjusted for age in 5 year periods, sex and level of education.
^{b}Adjusted for age in 5 year periods, sex, level of education and salbutamol and corticosteroids simultaneously.
RR: Rate ratio; PD: Parkinson's disease; CI: Confidence interval

Propranolol is used to treat essential tremor, which might be misdiagnosed as a first sign of PD. To reduce this source of bias, all individuals with an indication of essential tremor or other neurological diseases were excluded and only those with cardiovascular diagnoses were kept. Moreover, a time lag between time of first exposure to propranolol and PD onset was introduced. Using time lags of one and two years only slightly reduced the effect estimates (RR from 2.20 to 1.82). This made it unlikely for reverse causality to explain a major part of this association.

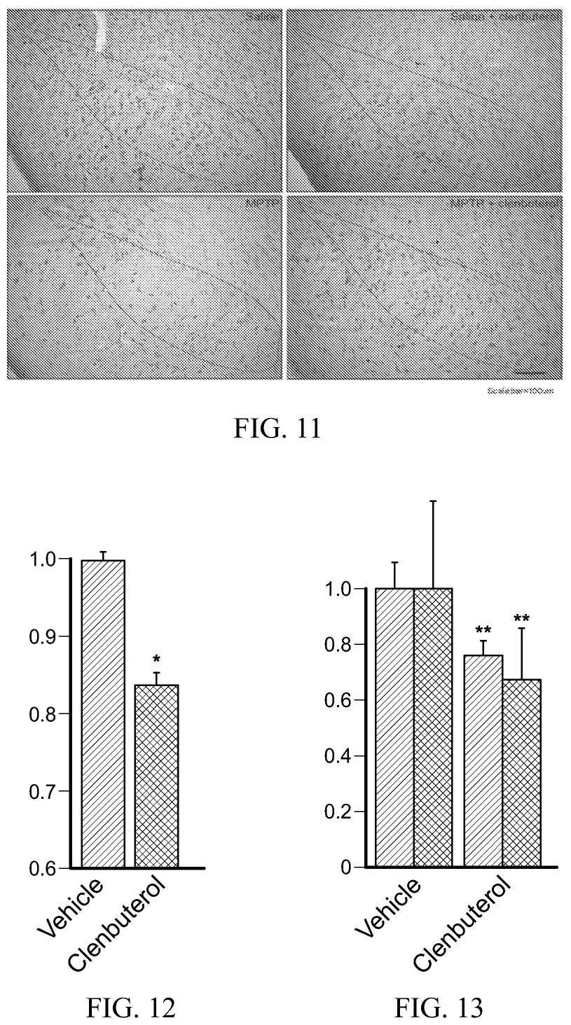

In addition to α-synuclein, chemicals such as N-methyl-4-phenyl-1,2,3,6-tetrahydropyridine (MPTP) (21, 22) and rotenone (23, 24) are implicated in the mechanism of sporadic PD. These chemicals inhibit the flow of electrons through complex I of the electron transport chain and foster buildup of superoxide and other reactive oxygen species particularly in dopamine neurons (22, 25, 26). It was determined whether clenbuterol treatment can protect against MPTP-induced degeneration of tyrosine hydroxylase (TH)-positive neurons in the substantia nigra pars compacta (SNpc) of a mouse model of PD (10, 22). Clenbuterol treatment abrogated the MPTP-induced loss of TH+ neurons (FIGS. 9C and D) and, importantly, also blocked the loss of cresyl-violet (CV) stained cells in the SNpc (FIG. 9E, FIG. 11).

Triplication of the SNCA locus causes autosomal dominant PD (1, 2) with iPSC-derived neurons constitutively over-expressing endogenous α-synuclein (27). Increased levels of wild-type α-synuclein cause mitochondrial impairment and an increase in superoxide and other reactive oxygen species (28, 29) possibly due to interference with mitochondrial protein import (30). Next, it was determined whether clenbuterol could be helpful towards normalizing SNCA expression levels in human iPSC-derived neuronal cells of a patient carrying the SNCA-triplication. SNCA-triplication iPSC-derived neuronal progenitor cells (NPCs) were treated with clenbuterol (20 μM) and endogenous α-synuclein protein and SNCA mRNA expression were significantly reduced (P<0.05 and <0.005, two-tailed t-test, respectively) (FIG. 9F). Similarly, SNCA expression was reduced in SNCA-triplication iPSC-derived neurons cultured for 8 weeks and then treated with clenbuterol (20 μM) for three days (FIG. 12).

Furthermore, Parkinson's patient-derived NPCs carrying the pathogenic SNCA locus triplication show increased mitochondrial superoxide production and reduced viability under exposure to the environmental mitochondrial complex I toxin rotenone (28). Clenbuterol treatment ameliorated this increased mitochondria associated superoxide production (FIG. 9G) and rescued viability (FIG. 9H) similar to partial SNCA knockdown (28).

Based on these results, a model in which β2AR antagonists is proposed to increase SNCA expression via H3K27 acetylation with α-synuclein accumulation, mitochondrial oxidative stress, dopaminergic neurodegeneration, and increased risk of PD. By contrast, β2AR agonists are expected to promote dopamine neuron health by reducing SNCA expression (via H3K27 deacetylation) and the production of mitochondrial free radicals. This could benefit nigral dopamine neurons prone to mitochondrial bioenergetics dysfunction even at early stages of Lewy body neuropathology (31) and preferentially vulnerable to mitochondrial complex I toxins (22).

### References

1. A. B. Singleton, M. Farrer, J. Johnson, A. Singleton, S. Hague, J. Kachergus, M. Hulihan, T. Peuralinna, A. Dutra, R. Nussbaum, S. Lincoln, A. Crawley, M. Hanson, D. Maraganore, C. Adler, M. R. Cookson, M. Muenter, M. Baptista, D. Miller, J. Blancato, J. Hardy, K. Gwinn-Hardy, alpha-Synuclein locus triplication causes Parkinson's disease. Science 302, 841 (2003).

2. M. Farrer, J. Kachergus, L. Forno, S. Lincoln, D. S. Wang, M. Hulihan, D. Maraganore, K. Gwinn-Hardy, Z. Wszolek, D. Dickson, J. W. Langston, Comparison of kindreds with parkinsonism and alpha-synuclein genomic multiplications. Annals of neurology 55, 174-179 (2004).

3. D. W. Miller, S. M. Hague, J. Clarimon, M. Baptista, K. Gwinn-Hardy, M. R. Cookson, A. B. Singleton, Alpha-synuclein in blood and brain from familial Parkinson disease with SNCA locus triplication. Neurology 62, 1835-1838 (2004).

4. L. Pihlstrom, M. Toft, Genetic variability in SNCA and Parkinson's disease. Neurogenetics 12, 283-293 (2011).

5. C. R. Scherzer, J. A. Grass, Z. Liao, I. Pepivani, B. Zheng, A. C. Eklund, P. A. Ney, J. Ng, M. McGoldrick, B. Mollenhauer, E. H. Bresnick, M. G. Schlossmacher, GATA transcription factors directly regulate the Parkinson's disease-linked gene alpha synuclein. Proceedings of the National Academy of Sciences of the United States of America 105, 10907-10912 (2008).

6. R. L. Clough, L. Stefanis, A novel pathway for transcriptional regulation of alpha synuclein. Faseb J 21, 596-607 (2007).

7. A. Jowaed, I. Schmitt, 0. Kaut, U. Wullner, Methylation regulates alpha-synuclein expression and is decreased in Parkinson's disease patients' brains. The Journal of neuroscience 30, 6355-6359 (2010).

8. E. Junn, K. W. Lee, B. S. Jeong, T. W. Chan, J. Y. Im, M. M. Mouradian, Repression of alpha-synuclein expression and toxicity by microRNA-7. Proc Natl Acad Sci USA 106, 13052-13057 (2009).

9. M. Carbone, S. Duty, M. Rattray, Riluzole neuroprotection in a Parkinson's disease model involves suppression of reactive astrocytosis but not GLT-1 regulation. BMC Neurosci 13, 38 (2012).

10. Supplementary Results and Methods are available online.

11. U. Dettmer, A. J. Newman, F. Soldner, E. S. Luth, N. C. Kim, V. E. von Saucken, J. B. Sanderson, R. Jaenisch, T. Bartels, D. Selkoe, Parkinson-causing alpha-synuclein missense mutations shift native tetramers to monomers as a mechanism for disease initiation. Nat Commun 6, 7314 (2015).

12. L. C. Gleeson, K. J. Ryan, E. W. Griffin, T. J. Connor, A. Harkin, The beta2-adrenoceptor agonist clenbuterol elicits neuroprotective, anti-inflammatory and neurotrophic actions in the kainic acid model of excitotoxicity. Brain Behav Immun 24, 1354-1361 (2010).

13. J. M. O'Donnell, Pharmacological characterization of the discriminative stimulus effects of clenbuterol in rats. Pharmacology, biochemistry, and behavior 58, 813-818 (1997).

14. S. Mu, T. Shimosawa, S. Ogura, H. Wang, Y. Uetake, F. Kawakami-Mori, T. Marumo, Y. Yatomi, D. S. Geller, H. Tanaka, T. Fujita, Epigenetic modulation of the renal betaadrenergic-WNK4 pathway in salt-sensitive hypertension. Nature medicine 17, 573-580 (2011).

15. Y. Leng, D. M. Chuang, Endogenous alpha-synuclein is induced by valproic acid through histone deacetylase inhibition and participates in neuroprotection against glutamate induced excitotoxicity. The Journal of neuroscience 26, 7502-7512 (2006).

16. The Norwegian Prescription Database. The Norwegian Institute of Public Health.(available on the world wide web at norpd.no).

17. G. Alves, B. Muller, K. Herlofson, I. HogenEsch, W. Telstad, D. Aarsland, O. B. Tysnes, J. P. Larsen, Incidence of Parkinson's disease in Norway: the Norwegian ParkWest study. J Neurol Neurosurg Psychiatry 80, 851-857 (2009).

18. B. Ritz, A. Ascherio, H. Checkoway, K. S. Marder, L. M. Nelson, W. A. Rocca, G. W. Ross, D. Strickland, S. K. Van Den Eeden, J. Gorell, Pooled analysis of tobacco use and risk of Parkinson disease. Archives of neurology 64, 990-997 (2007).

19. P. Subbarao, P. J. Mandhane, M. R. Sears, Asthma: epidemiology, etiology and risk factors. CMAJ 181, E181-190 (2009).

20. M. Lund, Social Inequality in Cigarette Consumption, Cigarette Dependence, and Intention to Quit among Norwegian Smokers. Biomed Res Int 2015, 835080 (2015).

21. J. W. Langston, P. Ballard, J. W. Tetrud, I. Irwin, Chronic Parkinsonism in humans due to a product of meperidine-analog synthesis. Science 219, 979-980 (1983).

22. W. Dauer, S. Przedborski, Parkinson's disease: mechanisms and models. Neuron 39, 889-909 (2003).

23. R. Betarbet, T. B. Sherer, G. MacKenzie, M. Garcia-Osuna, A. V. Panov, J. T. Greenamyre, Chronic systemic pesticide exposure reproduces features of Parkinson's disease. Nat Neurosci 3, 1301-1306 (2000).

24. C. M. Tanner, F. Kamel, G. W. Ross, J. A. Hoppin, S. M. Goldman, M. Korell, C. Marras, G. S. Bhudhikanok, M. Kasten, A. R. Chade, K. Comyns, M. B. Richards, C. Meng, B. Priestley, H. H. Fernandez, F. Cambi, D. M. Umbach, A. Blair, D. P. Sandler, J. W. Langston, Rotenone, paraquat, and Parkinson's disease. Environ Health Perspect 119, 866-872 (2011).

25. E. Hasegawa, D. Kang, K. Sakamoto, A. Mitsumoto, T. Nagano, S. Minakami, K. Takeshige, A dual effect of 1-methyl-4-phenylpyridinium (MPP+)-analogs on the respiratory chain of bovine heart mitochondria. Arch Biochem Biophys 337, 69-74 (1997).

26. E. Bezard, S. Dovero, B. Bioulac, C. Gross, Effects of different schedules of MPTP administration on dopaminergic neurodegeneration in mice. Exp Neurol 148, 288-292 (1997).

27. C. Y. Chung, V. Khurana, P. K. Auluck, D. F. Tardiff, J. R. Mazzulli, F. Soldner, V. Baru, Y. Lou, Y. Freyzon, S. Cho, A. E. Mungenast, J. Muffat, M. Mitalipova, M. D. Pluth, N. T. Jui, B. Schule, S. J. Lippard, L. H. Tsai, D. Krainc, S. L. Buchwald, R. Jaenisch, S. Lindquist, Identification and rescue of alpha-synuclein toxicity in Parkinson patient-derived neurons. Science 342, 983-987 (2013).

28. A. Flierl, L. M. Oliveira, L. J. Falomir-Lockhart, S. K. Mak, J. Hesley, F. Soldner, D. J. Arndt-Jovin, R. Jaenisch, J. W. Langston, T. M. Jovin, B. Schule, Higher vulnerability and stress sensitivity of neuronal precursor cells carrying an alpha-synuclein gene triplication. PLoS One 9, e112413 (2014).

29. L. J. Hsu, Y. Sagara, A. Arroyo, E. Rockenstein, A. Sisk, M. Mallory, J. Wong, T. Takenouchi, M. Hashimoto, E. Masliah, alpha-synuclein promotes mitochondrial deficit and oxidative stress. Am J Pathol 157, 401-410 (2000).

30. R. Di Maio, P. J. Barrett, E. K. Hoffman, C. W. Barrett, A. Zharikov, A. Borah, X. Hu, J. McCoy, C. T. Chu, E. A. Burton, T. G. Hastings, J. T. Greenamyre, alpha-Synuclein binds to TOM20 and inhibits mitochondrial protein import in Parkinson's disease. Sci Transl Med 8, 342ra378 (2016).

31. B. Zheng, Z. Liao, J. J. Locascio, K. A. Lesniak, S. S. Roderick, M. L. Watt, A. C. Eklund, Y. Zhang-James, P. D. Kim, M. A. Hauser, E. Grunblatt, L. B. Moran, S. A. Mandel, P. Riederer, R. M. Miller, H. J. Federoff, U. Wullner, S. Papapetropoulos, M. B. Youdim, I. Cantuti-Castelvetri, A. B. Young, J. M. Vance, R. L. Davis, J. C. Hedreen, C. H. Adler, T. G. Beach, M. B. Graeber, F. A. Middleton, J. C. Rochet, C. R. Scherzer, PGC-lalpha, a potential therapeutic target for early intervention in Parkinson's disease. Sci Transl Med 2, 52ra73 (2010).

32. T. C. Rainbow, B. Parsons, B. B. Wolfe, Quantitative autoradiography of beta 1- and beta 2-adrenergic receptors in rat brain. Proceedings of the National Academy of Sciences of the United States of America 81, 1585-1589 (1984).

33. L. Qian, H. M. Wu, S. H. Chen, D. Zhang, S. F. Ali, L. Peterson, B. Wilson, R. B. Lu, J. S. Hong, P. M. Flood. β2-adrenergic receptor activation prevents rodent dopaminergic neurotoxicity by inhibiting microglia via a novel signaling pathway. J Immunol. 186, 4443-4454 (2011).

34. GTEx Consortium, Human genomics. The Genotype-Tissue Expression (GTEx) pilot analysis: multitissue gene regulation in humans. Science 348, 648-660 (2015).

35. ENCODE Project Consortium, An integrated encyclopedia of DNA elements in the human genome. Nature 489, 57-74 (2012).

36. M. P. Mount, A. Lira, D. Grimes, P. D. Smith, S. Faucher, R. Slack, H. Anisman, S. Hayley, D. S. Park. Involvement of interferon-gamma in microglial-mediated loss of dopaminergic neurons. J Neurosci. 27, 3328-3337(2007).

37. H. Takahashi, T. Lassmann, M. Murata, P. Carninci, 5′ end-centered expression profiling using cap-analysis gene expression and next-generation sequencing. Nature protocols 7, 542-561 (2012).

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

1. A method of treating a subject who has a synucleinopathy, the method comprising:
administering to a subject in need of such treatment therapeutically effective amounts of a β2-adrenoreceptor agonist and at least one therapeutic agent selected from the group consisting of: a synucleinopathy therapeutic agent, a β2-adrenoreceptor antagonist, and a health supplement, wherein the health supplement is selected from the group consisting of caffeine, inosine, creatine, coenzyme Q 10, vitamin E, and omega-3 fatty acids, to thereby treat the synucleinopathy in the subject.

2. The method of claim 1, wherein the method further comprises identifying the subject as having a synucleinopathy, prior to administering.

3. The method of claim 1, wherein the method comprises administering a β2-adrenoreceptor agonist, a synucleinopathy therapeutic agent and at least one of the health supplements.

4. The method of claim 1, wherein the β2-adrenoreceptor agonist is a blood brain penetrant β2-adrenoreceptor agonist.

5. The method of claim 1, wherein the β2-adrenoreceptor agonist is selected from the group consisting of bitolterol, fenoterol, isoprenaline, levosalbutamol, orciprenaline, pirbuterol, procaterol, ritodrine, salbutamol, terbutaline, arformoterol, bambuterol, clenbuterol, formoterol, salmeterol, abediterol, carmoterol, indacaterol, olodaterol, vilanterol, metaproterenol, mabuterol, and zilpaterol.

6. The method of claim 1, wherein the β2-adrenoreceptor agonist is selected from the group consisting of metaproterenol, clenbuterol and salbutamol.

7. The method of claim 1, wherein the synucleinopathy therapeutic agent is selected from the group consisting of levodopa, carbidopa, entacapone, ropinirole, rotigotine, pramipexole, bromocriptine, rasagiline, selegiline, amantadine and trihexphenidyl.

8. The method of claim 1, wherein the method comprises administering a β2-adrenoreceptor agonist and a β2-adrenoreceptor antagonist.

9. The method of claim 8, wherein the β2-adrenoreceptor antagonist does not penetrate the blood brain barrier.

10. The method of claim 8, wherein the β2-adrenoreceptor antagonist is selected from the group consisting of carteolol, carvedilol, labetalol, nadolol, penbutolol, pindolol, sotalol, timolol, oxprenolol and butaxamine.

11. The method of claim 1, further comprising administering therapeutically effective amounts of riluzole hydrochloride, or a pharmaceutically acceptable salt, prodrug, or isomer thereof.

12. The method of claim 1, wherein the β2-adrenoreceptor agonist and the at least one therapeutic agent are administered simultaneously to the subject; wherein the β2-adrenoreceptor agonist is administered to the subject prior to administration of the at least one therapeutic agent; or wherein the at least one therapeutic agent is administered to the subject prior to administration of the β2-adrenoreceptor agonist.

13. The method of claim 1, wherein the subject has Parkinson's disease,

14. The method of claim 1, wherein the subject does not have Parkinson's disease.

15.-18. (canceled)
